SEQUENCE LISTING (86 sequences)

SEQ ID NO: 1 - DNA, 105 bp, synthetic construct
  source 1..105: mol_type = other DNA
gtacgacaactaccccataccaaaccttccttcgtacttaaaaatcccggccagattttgccaatcaccctatagtgagtcgtattagttagattttgccaatca

SEQ ID NO: 2 - DNA, 15 bp, synthetic construct
  source 1..15: mol_type = other DNA
ttggcaaaatctaac

SEQ ID NO: 3 - DNA, 20 bp, synthetic construct
  source 1..20: mol_type = other DNA
taatacgactcactataggg

SEQ ID NO: 4 - DNA, 16 bp, synthetic construct
  source 1..16: mol_type = other DNA
ttggcaaaatctaact

SEQ ID NO: 5 - DNA, 19 bp, synthetic construct
  source 1..19: mol_type = other DNA
aatacgactcactataggg

SEQ ID NO: 6 - DNA, 17 bp, synthetic construct
  source 1..17: mol_type = other DNA
ttggcaaaatctaacta

SEQ ID NO: 7 - DNA, 18 bp, synthetic construct
  source 1..18: mol_type = other DNA
atacgactcactataggg

SEQ ID NO: 8 - DNA, 18 bp, synthetic construct
  source 1..18: mol_type = other DNA
ttggcaaaatctaactaa

SEQ ID NO: 9 - DNA, 17 bp, synthetic construct
  source 1..17: mol_type = other DNA
tacgactcactataggg

SEQ ID NO: 10 - DNA, 19 bp, synthetic construct
  source 1..19: mol_type = other DNA
ttggcaaaatctaactaat

SEQ ID NO: 11 - DNA, 16 bp, synthetic construct
  source 1..16: mol_type = other DNA
acgactcactataggg

SEQ ID NO: 12 - DNA, 20 bp, synthetic construct
  source 1..20: mol_type = other DNA
ttggcaaaatctaactaata

SEQ ID NO: 13 - DNA, 15 bp, synthetic construct
  source 1..15: mol_type = other DNA
cgactcactataggg

SEQ ID NO: 14 - DNA, 16 bp, synthetic construct
  source 1..16: mol_type = other DNA
acgactcactataggg

SEQ ID NO: 15 - DNA, 72 bp, synthetic construct
  source 1..72: mol_type = other DNA
gtacgacaactaccccataccaaaccttccttcgtacccctatagtgagtcgtattatgccagccattctag

SEQ ID NO: 16 - DNA, 19 bp, synthetic construct
  source 1..19: mol_type = other DNA
cagcatcaccgccattaat

SEQ ID NO: 17 - DNA, 73 bp, synthetic construct
  source 1..73: mol_type = other DNA
gtacgacaactaccccataccaaaccttccttcgtacccctatagtgagtcgtattattgccagccattctag

SEQ ID NO: 18 - DNA, 20 bp, synthetic construct
  source 1..20: mol_type = other DNA
cagcatcaccgccatataat

SEQ ID NO: 19 - DNA, 74 bp, synthetic construct
  source 1..74: mol_type = other DNA
gtacgacaactaccccataccaaaccttccttcgtacccctatagtgagtcgtattatttgccagccattctag

SEQ ID NO: 20 - DNA, 21 bp, synthetic construct
  source 1..21: mol_type = other DNA
cagcatcaccgccataataat

SEQ ID NO: 21 - DNA, 75 bp, synthetic construct
  source 1..75: mol_type = other DNA
gtacgacaactaccccataccaaaccttccttcgtacccctatagtgagtcgtattattttgccagccattctag

SEQ ID NO: 22 - DNA, 22 bp, synthetic construct
  source 1..22: mol_type = other DNA
cagcatcaccgccataaataat

SEQ ID NO: 23 - DNA, 76 bp, synthetic construct
  source 1..76: mol_type = other DNA
gtacgacaactaccccataccaaaccttccttcgtacccctatagtgagtcgtattatttttgccagccattctag

SEQ ID NO: 24 - DNA, 23 bp, synthetic construct
  source 1..23: mol_type = other DNA
cagcatcaccgccataaaataat

SEQ ID NO: 25 - DNA, 75 bp, synthetic construct; identical to SEQ ID NO: 21
  source 1..75: mol_type = other DNA

SEQ ID NO: 26 - DNA, 22 bp, synthetic construct
  source 1..22: mol_type = other DNA
cagcatcaccgccattaataat

SEQ ID NO: 27 - DNA, 76 bp, synthetic construct; identical to SEQ ID NO: 23
  source 1..76: mol_type = other DNA

SEQ ID NO: 28 - DNA, 23 bp, synthetic construct
  source 1..23: mol_type = other DNA
cagcatcaccgccatttaataat

SEQ ID NO: 29 - DNA, 77 bp, synthetic construct
  source 1..77: mol_type = other DNA
gtacgacaactaccccataccaaaccttccttcgtacccctatagtgagtcgtattattttttgccagccattctag

SEQ ID NO: 30 - DNA, 24 bp, synthetic construct
  source 1..24: mol_type = other DNA
cagcatcaccgccattttaataat

SEQ ID NO: 31 - DNA, 78 bp, synthetic construct
  source 1..78: mol_type = other DNA
gtacgacaactaccccataccaaaccttccttcgtacccctatagtgagtcgtattatttttttgccagccattctag

SEQ ID NO: 32 - DNA, 25 bp, synthetic construct
  source 1..25: mol_type = other DNA
cagcatcaccgccatttttaataat

SEQ ID NO: 33 - DNA, 79 bp, synthetic construct
  source 1..79: mol_type = other DNA
gtacgacaactaccccataccaaaccttccttcgtacccctatagtgagtcgtattattttttttgccagccattctag

SEQ ID NO: 34 - DNA, 26 bp, synthetic construct
  source 1..26: mol_type = other DNA
cagcatcaccgccattttttaataat

SEQ ID NO: 35 - DNA, 74 bp, synthetic construct
  source 1..74: mol_type = other DNA
gtacgacaactaccccataccaaaccttccttcgtacccctatagtgagtcgtattattatgaggaacgagaag

SEQ ID NO: 36 - DNA, 21 bp, synthetic construct
  source 1..21: mol_type = other DNA
tgttgcgactacgtgaataat

SEQ ID NO: 37 - DNA, 74 bp, synthetic construct; identical to SEQ ID NO: 19
  source 1..74: mol_type = other DNA

SEQ ID NO: 38 - DNA, 21 bp, synthetic construct
  source 1..21: mol_type = other DNA
cagcatcaccgccataataat

SEQ ID NO: 39 - DNA, 74 bp, synthetic construct
  source 1..74: mol_type = other DNA
gtacgacaactaccccataccaaaccttccttcgtacccctatagtgagtcgtattattgctttagtggcagta

SEQ ID NO: 40 - DNA, 19 bp, synthetic construct
  source 1..19: mol_type = other DNA
tgtgttacattgtattaat

SEQ ID NO: 41 - DNA, 74 bp, synthetic construct
  source 1..74: mol_type = other DNA
gtacgacaactaccccataccaaaccttccttcgtacccctatagtgagtcgtattatttgttacattgtatgc

SEQ ID NO: 42 - DNA, 21 bp, synthetic construct
  source 1..21: mol_type = other DNA
tctgccgaaagcttgaataat

SEQ ID NO: 43 - DNA, 74 bp, synthetic construct
  source 1..74: mol_type = other DNA
gtacgacaactaccccataccaaaccttccttcgtacccctatagtgagtcgtattattagttcctggtcccca

SEQ ID NO: 44 - DNA, 21 bp, synthetic construct
  source 1..21: mol_type = other DNA
gttccttgtctgattaataat

SEQ ID NO: 45 - DNA, 75 bp, synthetic construct
  source 1..75: mol_type = other DNA
ggatccattcgttacctggctctcgccagtcgggatccccctatagtgagtcgtattattagttgacaccctgat

SEQ ID NO: 46 - DNA, 75 bp, synthetic construct
  source 1..75: mol_type = other DNA
ggatccattcgttacctggctctcgccagtcgggatccccctatagtgagtcgtattattagttaacaccctgat

SEQ ID NO: 47 - DNA, 75 bp, synthetic construct
  source 1..75: mol_type = other DNA
ggatccattcgttacctggctctcgccagtcgggatccccctatagtgagtcgtattattagtttacaccctgat

SEQ ID NO: 48 - DNA, 75 bp, synthetic construct
  source 1..75: mol_type = other DNA
ggatccattcgttacctggctctcgccagtcgggatccccctatagtgagtcgtattattagttcacaccctgat

SEQ ID NO: 49 - DNA, 21 bp, synthetic construct
  source 1..21: mol_type = other DNA
cagggacttctgtgcaataat

SEQ ID NO: 50 - DNA, 74 bp, synthetic construct
  source 1..74: mol_type = other DNA
gtacgacaactaccccataccaaaccttccttcgtacccctatagtgagtcgtattattcgggtaacgtcaatg

SEQ ID NO: 51 - DNA, 21 bp, synthetic construct
  source 1..21: mol_type = other DNA
ccggtgcttcttctgaataat

SEQ ID NO: 52 - DNA, 74 bp, synthetic construct
  source 1..74: mol_type = other DNA
gtacgacaactaccccataccaaaccttccttcgtacccctatagtgagtcgtattattccacgctttcgcacc

SEQ ID NO: 53 - DNA, 21 bp, synthetic construct
  source 1..21: mol_type = other DNA
aatcctgtttgctccaataat

SEQ ID NO: 54 - DNA, 74 bp, synthetic construct
  source 1..74: mol_type = other DNA
gtacgacaactaccccataccaaaccttccttcgtacccctatagtgagtcgtattattcgctttcgcacatca

SEQ ID NO: 55 - DNA, 21 bp, synthetic construct
  source 1..21: mol_type = other DNA
cctgtttgatccccaaataat

SEQ ID NO: 56 - DNA, 74 bp, synthetic construct
  source 1..74: mol_type = other DNA
gtacgacaactaccccataccaaaccttccttcgtacccctatagtgagtcgtattattggacttaacccaaca

SEQ ID NO: 57 - DNA, 21 bp, synthetic construct
  source 1..21: mol_type = other DNA
gttgcgctcgttgcgaataat

SEQ ID NO: 58 - DNA, 40 bp, synthetic construct
  source 1..40: mol_type = other DNA
taatacgactcactataggggcagtcaagcctcttctcgt

SEQ ID NO: 59 - DNA, 20 bp, synthetic construct
  source 1..20: mol_type = other DNA
aacattggccgcaaattgca

SEQ ID NO: 60 - DNA, 40 bp, synthetic construct
  source 1..40: mol_type = other DNA
taatacgactcactataggggcagtcaagcctcttctcgc

SEQ ID NO: 61 - DNA, 20 bp, synthetic construct
  source 1..20: mol_type = other DNA
ggtgtgacttccatgccaat

SEQ ID NO: 62 - DNA, 40 bp, synthetic construct
  source 1..40: mol_type = other DNA
taatacgactcactatagggatagtcaatcatcttcaaga

SEQ ID NO: 63 - DNA, 20 bp, synthetic construct
  source 1..20: mol_type = other DNA
ttctgatgggtaagtttaaa

SEQ ID NO: 64 - DNA, 40 bp, synthetic construct
  source 1..40: mol_type = other DNA
taatacgactcactataggggtgctccttcccggtctcag

SEQ ID NO: 65 - DNA, 20 bp, synthetic construct
  source 1..20: mol_type = other DNA
cttccaaagttgtggtcaag

SEQ ID NO: 66 - DNA, 40 bp, synthetic construct
  source 1..40: mol_type = other DNA
taatacgactcactataggggctctaataactcatctcgt

SEQ ID NO: 67 - DNA, 20 bp, synthetic construct
  source 1..20: mol_type = other DNA
tcccccatattgtgattaaa

SEQ ID NO: 68 - DNA, 40 bp, synthetic construct
  source 1..40: mol_type = other DNA
taatacgactcactatagggctgctcctaattccagatct

SEQ ID NO: 69 - DNA, 20 bp, synthetic construct
  source 1..20: mol_type = other DNA
aactctaatcttgatccaaa

SEQ ID NO: 70 - DNA, 40 bp, synthetic construct
  source 1..40: mol_type = other DNA
taatacgactcactatagggctgcttctaatagtcgacca

SEQ ID NO: 71 - DNA, 20 bp, synthetic construct
  source 1..20: mol_type = other DNA
aagtctaatttagaaccaaa

SEQ ID NO: 72 - DNA, 70 bp, synthetic construct
  source 1..70: mol_type = other DNA
taatacgactcactatagggtctaaccaggttgctgttctttatcagggtgttaactgcacagaagtccc

SEQ ID NO: 73 - DNA, 50 bp, synthetic construct
  source 1..50: mol_type = other DNA
ggagtaagttgatctgcatgaatagcaacagggacttctgtgcagttaac

SEQ ID NO: 74 - DNA, 70 bp, synthetic construct
  source 1..70: mol_type = other DNA
taatacgactcactatagggtctaaccaggttgctgttctttatcaggatgttaactgcacagaagtccc

SEQ ID NO: 75 - DNA, 50 bp, synthetic construct; identical to SEQ ID NO: 73
  source 1..50: mol_type = other DNA

SEQ ID NO: 76 - RNA, 380 nt, synthetic construct
  source 1..380: mol_type = other RNA
gcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgca

SEQ ID NO: 77 - RNA, 442 nt, synthetic construct
  source 1..442: mol_type = other RNA
gcagtcaagcctcttctcgctcctcatcacgtagtcgcggtaattcaagaaattcaactcctggcagcagtaggggaaattctcctgctcgaatggctagcggaggtggtgaaactgccctcgcgctattgctgctagacagattgaaccagcttgagagcaaagtttctggtaaaggccaacaacaacaaggccaaactgtcactaagaaatctgctgctgaggcatctaaaaagcctcgccaaaaacgtactgccacaaaacagtacaacgtcactcaagcatttgggagacgtggtccagaacaaacccaaggaaatttcggggaccaagacctaatcagacaaggaactgattacaaacattggccgcaaattgcacaatttgctccaagtgcctctgcattctttggaatgtcacgcattggcatggaagtcacacc

SEQ ID NO: 78 - RNA, 451 nt, synthetic construct
  source 1..451: mol_type = other RNA
atagtcaatcatcttcaagagcctctagcttaagcagaaactcttccagatctagttcacaaggttcaagatcaggaaactctacccgcggcacttctccaggtccatctggaatcggagcagtaggaggtgatctactttaccttgatcttctgaacagactacaagcccttgagtctggcaaagtaaagcaatcgcagccaaaagtaatcactaagaaagatgctgctgctgctaaaaataagatgcgccacaagcgcacttccaccaaaagtttcaacatggtgcaagcttttggtcttcgcggaccaggagacctccagggaaactttggtgatcttcaattgaataaactcggcactgaggacccacgttggccccaaattgctgagcttgctcctacagccagtgcttttatgggtatgtcgcaatttaaacttacccatcagaa

SEQ ID NO: 79 - RNA, 391 nt, synthetic construct
  source 1..391: mol_type = other RNA
gtgctccttcccggtctcagtcgaggtcgcagagtcgcggtcgtggtgaatccaaacctcaatctcggaatccttcaagtgacagaaaccataacagtcaggatgacatcatgaaggcagttgctgcggctcttaaatctttaggttttgacaagcctcaggaaaaagataaaaagtcagcgaaaacgggtactcctaagccttctcgtaatcagagtcctgcttcttctcaaacttctgccaagagtcttgctcgttctcagagttctgaaacaaaagaacaaaagcatgaaatgcaaaagccacggtggaaaagacagcctaatgatgatgtgacatctaatgtcacacaatgttttggccccagagaccttgaccacaactttggaag

SEQ ID NO: 80 - RNA, 355 nt, synthetic construct
  source 1..355: mol_type = other RNA
gctctaataactcatctcgtgctagcagtcgttcttcaactcgtaacaactcacgagactcttctcgtagcacttcaagacaacagtctcgcactcgttctgattctaaccagtcttcttcagatcttgttgctgctgttactttggctttaaagaacttaggttttgataaccagtcgaagtcacctagttcttctggtacttccactcctaagaaacctaataagcctctttctcaacccagggctgataagccttctcagttgaagaaacctcgttggaagcgtgttcctaccagagaggaaaatgttattcagtgctttggtcctcgtgattttaatcacaatatggggga

SEQ ID NO: 81 - RNA, 412 nt, synthetic construct
  source 1..412: mol_type = other RNA
ctgctcctaattccagatctacttcgcgcacatccagcagagcctctagtgcaggatcgcgtagtagagccaattctggcaatagaacccctacctctggtgtaacacctgacatggctgatcaaattgctagtcttgttctggcaaaacttggcaaggatgccactaaacctcagcaagtaactaagcatactgccaaagaagtcagacagaaaattttgaataagccccgccagaagaggagccccaataaacaatgcactgttcagcagtgttttggtaagagaggccctaatcagaattttggtggtggagaaatgttaaaacttggaactagtgacccacagttccccattcttgcagaactcgcacccacagctggtgcgtttttctttggatcaagattagagtt

SEQ ID NO: 82 - RNA, 409 nt, synthetic construct
  source 1..409: mol_type = other RNA
ctgcttctaatagtcgaccaggttcacgttctcaatcacgtggacccaataatcgttcattaagtagaagtaattctaattttagacattcagattctatagtaaaacctgatatggctgatgagatcgctaatcttgttttagccaagcttggtaaagattctaaacctcagcaagtcactaagcaaaatgccaaggaaatcaggcataaaattttaacaaaacctcgccaaaagcgaactcctaataaacattgtaatgttcaacagtgttttggtaaaagaggaccttctcaaaattttggtaatgctgaaatgttaaagcttggtactaatgatcctcagtttcctattcttgcagaattagctcctacaccaggtgcttttttctttggttctaaattagactt

SEQ ID NO: 83 - RNA, 80 nt, synthetic construct
  source 1..80: mol_type = other RNA
tctaaccaggttgctgttctttatcagggtgttaactgcacagaagtccctgttgctattcatgcagatcaacttactcc

SEQ ID NO: 84 - RNA, 80 nt, synthetic construct
  source 1..80: mol_type = other RNA
tctaaccaggttgctgttctttatcaggatgttaactgcacagaagtccctgttgctattcatgcagatcaacttactcc

SEQ ID NO: 85 - RNA, 610 nt, synthetic construct
  variation 66: note = n is a, c, g, or u
  variation 526..527: note = n is a, c, g, or u
  source 1..610: mol_type = other RNA
agactcctacgggaggcagcagtggggaatattgcacaatgggcgcaagcctgatgcagccatgcngcgtgtatgaagaaggccttcgggttgtaaagtactttcagcggggaggaagggagtaaagttaatacctttgctcattgacgttacccgcagaagaagcaccggctaactccgtgccagcagccgcggtaatacggagggtgcaagcgttaatcggaattactgggcgtaaagcgcacgcaggcggtttgttaagtcagatgtgaaatccccgggctcaacctgggaactgcatctgatactggcaagcttgagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtccacgccgtaaacgatgtcgacttggaggttgtgcccttgaggcgtggcttccgganntaacgcgttaagtcgaccgcctggggagtacggccgcaaggttaaaactcaaatgaattgacgggggccgcacaagcggtgga

SEQ ID NO: 86 - RNA, 524 nt, synthetic construct
  source 1..524: mol_type = other RNA
cagaagaggaaagtggaattccatgtgtagcggtgaaatgcgcagagatatggaggaacaccagtggcgaaggcgactttctggtctgtaactgacgctgatgtgcgaaagcgtggggatcaaacaggattagataccctggtagtccacgccgtaaacgatgagtgctaagtgttagggggtttcccgccccttagtgctgcagctaacgcattaagcactccgcctggggagtacgaccgcaaggttgaaactcaaaggaattgacggggacccgcacaagcggtggagcatgtggtttaattcgaagcaacgcgaagaaccttaccaaatcttgacatcctttgacaactctagagatagagccttccccttcgggggacaaagtgacaggtggtgcatggttgtcgtcagctcgtgtcgtgagatgttgggttaagtcccgcaacgagcgcaacccttaagcttagttgccatcattaagttgggcactctaagttgactgccggtgacaaaccggagga

(19) United States
(12) Patent Application Publication
(10) Pub. No.: US 2026/0152807 A1
(43) Pub. Date: Jun. 4, 2026

(54) PROBE SET FOR ISOTHERMAL SINGLE REACTION USING SPLIT T7 PROMOTER, AND USE THEREOF

(71) Applicant: KONKUK UNIVERSITY INDUSTRIAL COOPERATION CORP, Seoul (KR)

(72) Inventors: Ki Soo PARK, Seoul (KR); Tae Hwi YOON, Seoul (KR); Ji Ye SHIN, Gunpo-si, Gyeonggi-do (KR)

(73) Assignee: KONKUK UNIVERSITY INDUSTRIAL COOPERATION CORP, Seoul (KR)

(21) Appl. No.: 18/706,577

(22) PCT Filed: Nov. 1, 2022
(86) PCT No.: PCT/KR2022/016927

§ 371 Date: Jul. 8, 2024

(30) Foreign Application Priority Data
Nov. 1, 2021 (KR) 10-2021-0147950
Nov. 1, 2021 (KR) 10-2021-0147951

(51) Int. Cl. C12Q 1/689; C12Q 1/48; C12Q 1/686; C12Q 1/70

(52) U.S. Cl. CPC C12Q 1/689; C12Q 1/48; C12Q 1/686; C12Q 1/701; C12Y 207/07; C12Q 2600/156; C12Q 2600/16

(57) ABSTRACT

The present invention relates to a probe set for an isothermal single reaction using a split T7 promoter and a use thereof. In the present invention, a design is made to introduce a split T7 promoter into a 3-way junction structure to produce a large quantity of fluorescent RNA aptamers at an isothermal temperature in the presence of a target molecule, and a nucleic acid biomarker can be detected rapidly and conveniently with only one enzyme in one pot. The probe set allows for various applications through high-sensitivity detection of multiple nucleic acid biomarkers and detection in which analysis is performed without an additional nucleic acid extraction process. In one pot, multiplex analysis can be conducted and molecular diagnosis can be made of various entities including viruses and pathogens.

### CROSS REFERENCE TO RELATED APPLICATIONS

This application is a 35 U.S.C. § 371 National Stage of International Patent Application No. PCT/KR2022/016927, filed Nov. 1, 2022, designating the United States and claiming priority benefit from Korean Patent Application No. 10-2021-0147950, filed Nov. 1, 2021, and Korean Patent Application No. 10-2021-0147951, filed Nov. 1, 2021, the disclosures of which are incorporated herein in their entirety by reference, and priority is claimed to each of the foregoing.

### REFERENCE TO SEQUENCE LISTING

The instant application contains a Sequence Listing which has been submitted electronically in XML format and is hereby incorporated by reference in its entirety. Said XML copy, created on Jul. 29, 2025 is named “4669-224.xml” and is 86,016 bytes in size.

### TECHNICAL FIELD

The present technology relates to a system for quickly and conveniently detecting various target molecules, and the system was designed to produce a large amount of fluorescent RNA aptamers in the presence of a target molecule at an isothermal temperature by introducing a new split T7 promoter into a three-way junction structure. Accordingly, the limitations of existing on-site diagnosis may be overcome, and various applications are possible through high-sensitivity detection of multiple target molecules and detection in which analysis is performed without an additional extraction process.

Meanwhile, the present invention was supported by the following national research and development project.

[National Research and Development Project that Supported this Invention]
[Project serial number]1711157003
[Project number]2020R1C1C1012275
[Name of Ministry] Ministry of Science and ICT
[Name of project management (specialized) organization] National Research Foundation of Korea
[Research program name] Individual basic research (Ministry of Science and ICT) (R&D)
[Research project name] Exosome SELEX technology (E-SELEX) and discovery of cancer diagnostic biomarkers and development of ultra-sensitive detection system using the same
[Contribution rate]1/1
[Name of organization carrying out project] Konkuk University Industry-Academic Cooperation Foundation (Seoul)
[Research period] Mar. 1, 2022 to Feb. 28, 2023

### BACKGROUND

In December 2019, a new respiratory infectious RNA virus, severe acute respiratory syndrome coronavirus (SARS-CoV-2), and its mutant viruses emerged and rapidly spread around the world. Therefore, there is a need for a technology to quickly and conveniently detect SARS-CoV-2 to suppress the spread of the virus.

Currently, the most commonly used method for detecting SARS-CoV-2 is the quantitative reverse transcription polymerase chain reaction (qRT-PCR) based on nucleic acid amplification. The method has advantages that various primer-probe sets have been identified and it has high sensitive detection limits, but the method also has disadvantages that all PCR tests require a temperature control device and are performed in specialized laboratories, so they take a long time (one or two days) and are expensive.

To compensate for the shortcomings of the temperature control-based method and improve testing convenience, isothermal nucleic acid amplification methods using various strand displacement polymerases have been developed. The isothermal amplification methods were developed to compensate for the shortcomings of temperature control-based methods and to facilitate point-of-care testing. However, most isothermal amplification methods require a denaturation/annealing step through temperature control before starting the reaction. In addition, there is a disadvantage that the reactions to various enzymes are difficult to optimize, and the detection step is complicated. In particular, in the case of SARS-CoV-2, most isothermal amplification methods are carried out by converting RNA into cDNA, and since DNA amplification products are produced in a large amount, the converted cDNA and amplified DNA are not easily degraded and thus are vulnerable to contamination. Such contamination may interfere with the reactions and result in a high false positive rate. Therefore, a rapid single-step isothermal amplification method is still needed to detect SARS-CoV-2.

Signal-mediated amplification of RNA technology (SMART) based on a three-way junction structure may be applied to genomic DNA (gDNA), total RNA, etc. and has been used for highly sensitive detection of various nucleic acid biomarkers. However, despite the excellent advantages of SMART technology, it has the limitation that two enzymes, DNA polymerase and RNA polymerase, are required, and when the two enzymes coexist, a non-specific signal is generated, making a one-step reaction impossible, and it takes a long reaction time.

Meanwhile, fluorescent RNA aptamers have high specificity and affinity for specific molecules and exhibit a high fluorescence signal when bound. Because they have higher thermal and chemical stability compared to antibody molecules, which are proteins, fluorescent RNA aptamers are widely used in not only intracellular experiments but also extracellular experiments. These fluorescent RNA aptamers have the advantages that they are inexpensive, have low background signals, and may be applied to multiplex analysis.

Regarding SMART technology, International Patent Publication WO 99/37806 discloses a SMART technology for detecting a target nucleic acid sequence, but the target nucleic acid was detected using two polymerases, and a technology in which only one polymerase is used has not been disclosed.

Accordingly, the present inventors completed the present invention by focusing on target detection using a three-way junction structure that is highly specific and capable of easily distinguishing changes in a target molecule while using a single enzyme.

### SUMMARY OF INVENTION

Accordingly, the present inventors took efforts to develop a novel isothermal nucleic acid amplification technology that can quickly detect a target molecule while overcoming the shortcomings of existing SMART analysis, and as a result, they invented a novel isothermal nucleic acid amplification technology (37° C.) based on a three-way junction structure and inexpensively and easily designed a probe to allow for multiple analysis without additional labeling.

Therefore, one object of the present invention is to provide an isothermal one-pot reaction probe set for detecting one or more target molecules, including a first probe and a second probe.

Another object of the present invention is to provide a composition for detecting a target molecule, including the isothermal one-pot reaction probe set for detecting a target molecule including a first probe and a second probe.

Still another object of the present invention is to provide a kit for detecting a target molecule, including the composition for detecting a target molecule, a polymerase, and an isothermal one-pot reaction solution.

Yet another object of the present invention is to provide a method of detecting a target molecule, using the isothermal one-pot reaction probe set for detecting a target molecule.

Yet another object of the present invention is to provide an on-site molecular diagnostic method using the isothermal one-pot reaction probe set for detecting a target molecule.

Yet another object of the present invention is to provide an isothermal one-pot reaction probe set for detecting SARS-CoV-2 and/or detecting a mutant thereof, including a third probe and a fourth probe.

Yet another object of the present invention is to provide a composition for detecting SARS-CoV-2 and/or detecting a mutant thereof, including the probe set.

Yet another object of the present invention is to provide a kit for detecting SARS-CoV-2 and/or detecting a mutant thereof, including the composition for detecting SARS-CoV-2 and/or detecting a mutant thereof, a polymerase, and an isothermal one-pot reaction solution.

Yet another object of the present invention is to provide a method of detecting SARS-CoV-2 and/or detecting a mutant thereof, using the probe set for detecting SARS-CoV-2 and/or detecting a mutant thereof.

Yet another object of the present invention is to provide an on-site molecular diagnostic method using an isothermal one-pot reaction probe set for detecting SARS-CoV-2 and/or detecting a mutant thereof.

The terminology used in the present invention is for the purpose of describing particular embodiments only and is not intended to limit the present invention. Singular forms include plural forms, unless the context clearly indicates otherwise. In the present invention, terms such as “comprise” or “have” should be understood as specifying the presence of features, steps, operations, components, parts or combinations thereof described in the specification, not precluding the possibility of the presence or addition of one or more other features, steps, operations, components, parts or combinations thereof. Unless otherwise defined, all terms, including technical and scientific terms used herein, have the same meaning as generally understood by one of ordinary skill in the art to which the present invention pertains. Terms, such as those defined in commonly used dictionaries, should be interpreted as having a meaning that is consistent with their meaning in the context of the relevant art and are not interpreted in an idealized or overly formal sense unless clearly so defined in the present invention.

To achieve the above objects, the present invention provides an isothermal one-pot reaction probe set for detecting one or more target molecules, including a first probe and a second probe,
wherein the first probe is a promoter probe (PP) having a structure including General Formulas I and II below,

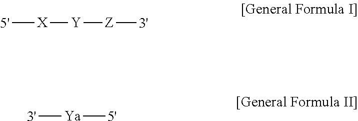

wherein in General Formula I, X is an aptamer sequence portion having an interactive labeling system including one label or multiple labels generating a detectable signal;
Y is a sequence complementary to a T7 promoter;
Z is a portion that binds or connects to a target molecule; and
X and Y are deoxyribonucleotides; and
in General Formula II, Ya is a partial sequence of the T7 promoter; and Ya is a deoxyribonucleotide;
wherein the second probe is a PP having a structure of General Formula III below,

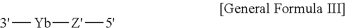

wherein in General Formula III,
Yb is absent or a partial sequence of the T7 promoter and is a deoxyribonucleotide; and
Z′ is a portion that binds or connects to a target molecule;
wherein the first probe and the second probe bind or connect to a target molecule and then transcription is initiated by a polymerase to generate a signal.

In one embodiment of the present invention, the target molecule may be one or more selected from the group consisting of a nucleic acid, a protein, a cell, and adenosine triphosphate (ATP).

In one embodiment of the present invention, when the target molecule is a nucleic acid, Z and Z′ may be nucleic acid sequences that complementarily bind to a nucleic acid to be detected, when the target molecule is a protein, Z and Z′ may be antibodies or aptamers that are capable of binding to the protein, when the target molecule is a cell, Z and Z′ may be antibodies or aptamers that are capable of binding to one or more selected from the group consisting of extracellular proteins, cellular phospholipids, bacterial peptidoglycans, and lipopolysaccharide (LPS), and when the target molecule is ATP, Z and Z′ may be split aptamers that are capable of binding to ATP.

In one embodiment of the present invention, the Ya and Yb may be partial sequences of a T7 promoter, and when the Ya and Yb are sequentially arranged in the order of Ya and Yb, they may form the T7 promoter, and a Ya:Yb split ratio may be 20:0 to 15:5.

In one preferred embodiment of the present invention, the Ya:Yb split ratio may be 16:4.

In one embodiment of the present invention, the label may be selected from the group consisting of a chemical label, an enzymatic label, a radioactive label, a fluorescent label, a luminescent label, a chemiluminescent label, and a metallic label.

In one embodiment of the present invention, General Formula I of the first probe may be modified into General Formula I′ further including an overlapping sequence W between Y and Z;

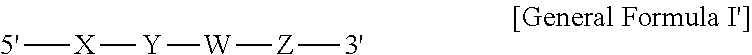

wherein General Formula III of the second probe may be modified into General Formula III′, further including an overlapping sequence W′ between Yb and Z′;

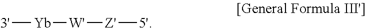

In one embodiment of the present invention, the length of overlapping sequences W and W′ may be 1 bp or 2 bp.

In one embodiment of the present invention, the isothermal one-pot reaction may be performed at a constant temperature in a range of 15° C. to 50° C.

In one embodiment of the present invention, the probe set may detect circular RNA separately from linear RNA.

In one embodiment of the present invention, the polymerase may be selected from the group consisting of bacteriophage T7 RNA polymerase, bacteriophage T3 polymerase, bacteriophage RNA polymerase, bacteriophage ΦII polymerase, Salmonella bacteriophage sp6 polymerase, Pseudomonas bacteriophage gh-1 polymerase, E. coli RNA polymerase holoenzyme, E. coli RNA polymerase core enzyme, human RNA polymerase I, human RNA polymerase II, human RNA polymerase III, human mitochondrial RNA polymerase, and variants thereof.

In one embodiment of the present invention, the isothermal one-pot reaction may be performed in a unified and simultaneous manner with a one-pot reaction solution containing Tris-HCl, MgCl₂, dithiothreitol (DTT), spermidine, ribonucleotide triphosphates (rNTPs), an RNase inhibitor, and a single-stranded DNA binding protein (SSB).

The present invention also provides a composition for detecting a target molecule, including the isothermal one-pot reaction probe set for detecting a target molecule.

In one embodiment of the present invention, the composition may include two or more types of isothermal one-pot reaction probe sets for detecting two or more types of target molecules.

In one embodiment of the present invention, the two or more types of isothermal one-pot reaction probe sets for detecting target molecules may each include different interactive labeling systems, and the two or more types of probe sets may respectively bind to different target molecules to enable multiplex detection of different target molecules.

In one embodiment of the present invention, the isothermal one-pot reaction may be performed at a constant temperature in a range of 15° C. to 50° C.

The present invention also provides a kit for detecting a target molecule, including the composition for detecting a target molecule, a polymerase, and an isothermal one-pot reaction solution.

In addition, the present invention provides a method of detecting a target molecule, using the isothermal one-pot reaction probe set for detecting a target molecule.

In one embodiment of the present invention, a step of performing an isothermal nucleic acid amplification reaction may be included prior to the method of detecting the target molecule.

In one embodiment of the present invention, the isothermal nucleic acid amplification reaction may be recombinase polymerase amplification (RPA).

The present invention also provides an on-site molecular diagnostic method using the isothermal one-pot reaction probe set for detecting a target nucleic acid sequence.

In one embodiment of the present invention, the molecular diagnostic method may be used in pathogenic microorganism detection.

In one embodiment of the present invention, the pathogenic microorganisms may be one or more selected from the group consisting of Staphylococcus Aureus, Vibrio vulnificus, E. coli, Middle East respiratory syndrome coronavirus, influenza A virus, severe acute respiratory syndrome coronavirus, respiratory syncytial virus (RSV), human immunodeficiency virus (HIV), herpes simplex virus (HSV), human papilloma virus (HPV), human parainfluenza viruses (HPIV), dengue virus, hepatitis B virus (HBV), yellow fever virus, rabies virus, Plasmodium, cytomegalovirus (CMV), Mycobacterium tuberculosis, Chlamydia trachomatis, rotavirus, human metapneumovirus (hMPV), Crimean-Congo hemorrhagic fever virus, Ebola virus, Zika virus, henipavirus, norovirus, Lassa virus, rhinovirus, flavivirus, Rift Valley fever virus, hand-foot-mouth disease virus, Salmonella sp., Shigella sp., Enterobacteriaceae sp., Pseudomonas sp., Moraxella sp., Helicobacter sp., and Stenotrophomonas sp.

The present invention provides an isothermal one-pot reaction probe set for detecting SARS-CoV-2 and/or detecting a mutant thereof, including a third probe and a fourth probe,
wherein the third probe is a PP having a structure including General Formulas IV and V below,

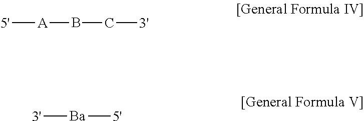

wherein in General Formula IV, A is an aptamer sequence portion having an interactive labeling system including one label or multiple labels generating a detectable signal;
B is a sequence complementary to a T7 promoter;
C is an upstream hybridization sequence (UHS) portion having a hybridization sequence complementary to a nucleic acid sequence of SARS-CoV-2 and/or a mutant thereof; the nucleic acid sequence of SARS-CoV-2 and/or a mutant thereof is DNA or RNA; and
A, B and C are deoxyribonucleotides; and
in General Formula V, Ba is a partial sequence of a T7 promoter; and Ba is a deoxyribonucleotide;
wherein the fourth probe is a PP having a structure consisting of General Formula VI,

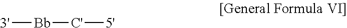

wherein in General Formula VI, Bb is absent or a partial sequence of T7 promoter;
C′ is a downstream hybridization sequence (DHS) portion having a hybridization sequence complementary to a nucleic acid sequence of SARS-CoV-2 and/or a mutant thereof; the nucleic acid sequence of SARS-CoV-2 and/or a mutant thereof is DNA or RNA; and Bb and C′ are deoxyribonucleotides; wherein the third probe and the fourth probe are hybridized to a nucleic acid sequence of SARS-CoV-2 and/or a mutant thereof and then transcription is initiated by a polymerase to generate a signal.

In one embodiment of the present invention, the Ba and Bb may be partial sequences of a T7 promoter, and when Ba and Bb are sequentially arranged in the order of Ba and Bb, they may form the T7 promoter, and a Ba:Bb split ratio may be 20:0 to 15:5

In one preferred embodiment of the present invention, the Ba:Bb split ratio may be 16:4.

In one embodiment of the present invention, the label may be selected from the group consisting of a chemical label, an enzymatic label, a radioactive label, a fluorescent label, a luminescent label, a chemiluminescent label, and a metallic label.

In one embodiment of the present invention, General Formula IV of the third probe may be modified into General Formula IV′ further including an overlapping sequence D between B and C;

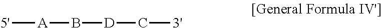

wherein General Formula VI of the fourth probe may be modified into General Formula VI′ further including an overlapping sequence D′ between Bb and C′;

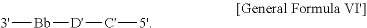

In one embodiment of the present invention, the length of overlapping sequences D and D′ may be 1 bp or 2 bp.

In one embodiment of the present invention, the isothermal one-pot reaction may be performed at a constant temperature in a range of 15° C. to 50° C.

In one embodiment of the present invention, the polymerase may be selected from the group consisting of bacteriophage T7 RNA polymerase, bacteriophage T3 polymerase, bacteriophage RNA polymerase, bacteriophage (DII polymerase, Salmonella bacteriophage sp6 polymerase, Pseudomonas bacteriophage gh-1 polymerase, E. coli RNA polymerase holoenzyme, E. coli RNA polymerase core enzyme, human RNA polymerase I, human RNA polymerase II, human RNA polymerase III, human mitochondrial RNA polymerase, and variants thereof.

In one embodiment of the present invention, the isothermal one-pot reaction may be performed in a unified and simultaneous manner with a one-pot reaction solution containing Tris-HCl, MgCl₂, DTT, spermidine, rNTPs, an RNase inhibitor, and an SSB.

In one embodiment of the present invention, in the isothermal one-pot reaction probe set for detecting SARS-CoV-2 and/or detecting a mutant thereof, a region of the nucleic acid sequence of SARS-CoV-2 and/or a mutant thereof may be an N gene or an S gene.

In one embodiment of the present invention, the isothermal one-pot reaction probe set for detecting SARS-CoV-2 may specifically bind to the N gene and may include at least one probe set selected from the group consisting of a probe set in which the third probe is SEQ ID NO: 37 and the fourth probe is SEQ ID NO: 38 and a probe set in which the third probe is SEQ ID NO: 41 and the fourth probe is SEQ ID NO: 42.

In one embodiment of the present invention, the isothermal one-pot reaction probe set for detecting a mutant may specifically bind to the S gene and may include a probe set in which the third probe is SEQ ID NO: 45 and the fourth probe is SEQ ID NO: 49.

The present invention provides a composition for detecting SARS-CoV-2 and/or detecting a mutant thereof, including the probe set.

The present invention provides a kit for detecting SARS-CoV-2 and/or detecting a mutant thereof, including the composition for detecting SARS-CoV-2 and/or detecting a mutant thereof of claim 36, a polymerase, and an isothermal one-pot reaction solution.

In addition, the present invention provides a method of detecting SARS-CoV-2 and/or detecting a mutant thereof, using a probe set for detecting SARS-CoV-2 and/or detecting a mutant thereof.

In one embodiment of the present invention, a step of performing an isothermal nucleic acid amplification reaction may be included prior to the method of detecting SARS-CoV-2 and/or detecting a mutant thereof.

In one embodiment of the present invention, the isothermal nucleic acid amplification reaction may be RPA.

The present invention also provides an on-site molecular diagnostic method under isothermal one-pot reaction conditions, using the isothermal one-pot reaction probe set for detecting SARS-CoV-2 and/or detecting a mutant thereof.

### Advantageous Effects

According to the present invention, a target molecule can be detected rapidly and conveniently with only one enzyme in one tube. In addition, the present invention provides an isothermal nucleic acid amplification method for which a probe can be conveniently designed without the need for additional labeling and which enables the implementation of a direct system through only thermal treatment without pretreatment. In addition, multiplex analysis can be conducted in one tube, and molecular diagnosis of viruses and pathogens is possible.

### BRIEF DESCRIPTION OF THE DRAWINGS

FIG. 1 shows a schematic diagram of split T7 promoter-based isothermal amplification with an RNA aptamer (hereinafter referred to as “STAR”) for detecting a target molecule. The three important components of the STAR reaction of the present invention are a STAR DNA probe, a T7 RNA polymerase, and a fluorescent dye. In the presence of target RNA, the STAR DNA probe forms a three-way junction structure, resulting in a double-stranded T7 promoter with a nick site. A T7 RNA polymerase may bind to a T7 promoter and initiate transcription to generate an RNA aptamer that allows a large amount of light to be emitted. Afterward, the RNA aptamer binds to a fluorescent dye to generate a highly enhanced fluorescence signal. A one-step reaction occurs within 30 minutes at 37° C.

FIG. 2 shows a schematic diagram of a case where a target molecule is a protein or cell.

FIG. 3 shows the results of splitting a split T7 promoter at various ratios.

FIG. 4 shows the results of optimizing an overlapping length.

FIG. 5 shows the results regarding the stability of a T7 promoter when a bulge structure is added to a three-way junction structure.

FIG. 6 shows the concentrations of DTT and MgCl₂ in a composition of a buffer for optimization of reaction conditions.

FIG. 7 shows the fluorescence intensity results measured to find the optimal concentrations of spermidine and single-stranded DNA binding protein (SSB).

FIGS. 8A-8D show fluorescence intensity results according to T7 RNA polymerase concentration, reaction time, reaction temperature, and cooling method.

FIG. 9 shows the results of comparing the transcription efficiency of a T7 promoter with a three-way spliced structure and a linear structure when an optimized buffer was used.

FIG. 10 shows the gel electrophoresis results for confirming a three-way junction structure.

FIG. 11 shows the fluorescence spectrum results without each element of STAR.

FIG. 12 shows a schematic diagram indicating that the method may be used to detect circular RNA separately from linear RNA.

FIG. 13 shows results indicating that the detection intensity increases when an isothermal nucleic acid amplification reaction is performed before performing the STAR reaction.

FIGS. 14A-14C relate to the detection sensitivity of each locus of the SARS-CoV-2 N gene: FIG. 14A shows a schematic diagram of a part of the SARS-CoV-2 N gene and the locus (1-5) of a target RNA; FIG. 14B shows a heatmap illustrating the detection sensitivity of STAR using five sets of DNA probes for different loci (L1-L5) of the N gene; and FIG. 14C shows the detection limit of STAR at L2 and L4 when the STAR probe set at each locus was 100 nM.

FIGS. 15A-15E show a combination of two sets of STAR probes to improve sensitivity: FIG. 15A shows a schematic diagram of two different loci of the SARS-CoV-2 N gene and of other human coronaviruses, in which the nucleotides of the RNA sequences of other viruses that are different from SARS-CoV-2 are shaded in gray; FIG. 15B shows an agarose gel electrophoresis image of the STAR probe set to the N gene RNA, in which the concentrations of the L2 and L4 probe sets are each 500 nM (a and b), and when the L2 and L4 probes are present simultaneously, the concentration of each probe set is 250 nM (c); FIG. 15C shows fluorescence signals measured in the presence of the L2 probe set (100 nM), the L4 probe set (100 nM), and both the L2 and L4 probe sets (50 nM+50 nM), with a target N gene RNA concentration of 1 nM; FIG. 15D shows the detection limit of using the STAR probe set combination at L2 and L4, in which the concentration of the STAR probe set is 50 nM at each different locus; and FIG. 15E shows the detection specificity of the STAR assay when using two STAR probe sets (L2 and L4), in which the RNA concentration of the SARS-CoV-2 N gene was 1 nM, that of the others was 100 nM, and the concentration of the START probe sets (L2 and L4) were each 50 nM.

FIGS. 16A-16D show the application of STAR for detecting a SARS-CoV-2 D614G mutation: FIG. 16A shows a schematic diagram of a STAR assay for a D614G mutation in the S gene of SARS-CoV-2, in which the mutation sites colored in yellow and a mismatch sequence colored in red was added to avoid false positive signals in the wild-type target; FIG. 16B shows malachite green (MG) signal intensity obtained after STAR using different nucleobases (A, T, G, and C) at the N4 position; FIG. 16C shows the detection limit of STAR for the D614G mutation, with the target RNA concentrations ranging from 1 nM to 100 fM; and FIG. 16D shows the results of detecting the D614G mutation in mixtures of mutant/wild targets at different molar ratios (0%, 0.1%, 1%, 10%, 50%, and 100%).

FIGS. 17A-17D show a system for multiplex detection: FIG. 17A shows a schematic diagram of a one-pot multiplex STAR for simultaneous detection of the SARS-CoV-2 N gene and D614G mutation; FIG. 17B shows normalized fluorescence emission spectra of TO1-biotin and MG binding to a mango aptamer and an MG aptamer, respectively; FIG. 17C shows a heatmap illustrating the results of one-pot multiplex detection of the N gene and D614G mutation with the target RNA concentration of 10 nM; and FIG. 17D shows the results of one-pot multiplex detection of the N gene and D614G mutation at different concentrations.

FIGS. 18A-18C show a system for pathogen detection: FIG. 18A shows a schematic diagram of pathogen detection using STAR with the STAR probes designed for the 16s rRNA region of the pathogen to detect the pathogen, in which (i) one is STAR using extracted total RNA and (ii) the other is direct STAR using a cell lysate obtained through thermal degradation; FIG. 18B shows the detection of bacterial 16S rRNA using STAR in which the total RNA concentrations ranging from 100 pg/μL to 100 fg/μL were tested; and FIG. 18C shows a heatmap illustrating the results of direct STAR for detecting different pathogens, in which the concentration of each pathogen was 10³ cells/μL.

### DETAILED DESCRIPTION

As described above, there is an emerging need for overcoming the disadvantages of an isothermal amplification reaction that even when two enzymes are used, a non-specific signal is generated, a one-step reaction is impossible, and a long reaction time is required. As a result of efforts by the present inventors to develop a novel isothermal nucleic acid amplification technology that can quickly detect a target molecule while overcoming the shortcomings of existing SMART analysis, they invented a novel isothermal nucleic acid amplification technology based on a three-way junction structure, which enables rapid analysis of a target molecule. In the present invention, the invention was designed so that a transcription reaction by a target substance occurs effectively through the control of the split ratio of a T7 promoter sequence, and based on this, a T7 promoter based on the three-way junction structure is formed only when the target molecule is present, through which a signal amplified by only one polymerase may be obtained in one tube. In addition, to further increase sensitivity, an additional three-way junction structure was designed to allow binding to multiple regions in a target molecule, and through this, an increase in sensitivity was achieved. In the present invention, when a target molecule is a nucleic acid sequence, changes in the sequence may be easily distinguished in a very specific manner, and a dependent sequence of a target molecule is amplified, rather than amplifying the target molecule as is, and based on this, two different types of light-up RNA aptamers were designed to implement multiplex analysis that can simultaneously identify two or more types of molecules in one tube. In addition, based on the excellence of the present invention, the present invention was applied to the detection of various pathogens, thereby verifying a method for successfully detecting the nucleic acid biomarkers of pathogenic microorganisms without an additional nucleic acid extraction process. Therefore, the present invention is excellent because detection may be performed simpler, faster, and more sensitively compared to the existing method.

Hereinafter, the present invention will be described in more detail.

The present invention provides an isothermal one-pot reaction probe set for detecting one or more target molecules, including a first probe and a second probe,
wherein the first probe is a promoter probe (PP) having a structure including General Formulas I and II below,

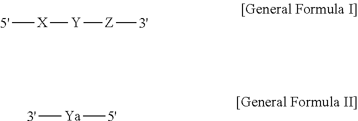

wherein in General Formula I, X is an aptamer sequence portion having an interactive labeling system including one label or multiple labels generating a detectable signal;
Y is a sequence complementary to a T7 promoter;
Z is a portion that binds or connects to a target molecule; and
X and Y are deoxyribonucleotides; and
in General Formula II, Ya is a partial sequence of the T7 promoter; and Ya is a deoxyribonucleotide;
wherein the second probe is a PP having a structure of General Formula III below,

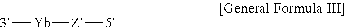

wherein in General Formula III,
Yb is absent or a partial sequence of the T7 promoter and is a deoxyribonucleotide; and
Z′ is a portion that binds or connects to a target molecule;
wherein the first probe and the second probe bind or connect to a target molecule and then transcription is initiated by a polymerase to generate a signal.

The first probe of the probe set of the present invention includes a structure including the following three different portions within one oligonucleotide molecule, and another oligonucleotide molecule: X, which is an aptamer sequence portion having an interactive labeling system including one label or multiple labels generating a detectable signal; Y, which is a sequence complementary to a T7 promoter; and Z, which is a portion that binds or connects to a target molecule; and Ya is a partial sequence of a T7 promoter complementary to Y.

In addition, the second probe of the probe set of the present invention also has a structure including one or two unique different portions within one oligonucleotide molecule: Yb, which is complementary to Y and a partial sequence of a T7 promoter and has a contiguous sequence with Ya; and Z′, which is a portion that binds or connects to a target molecule. Yb may not be present when a split ratio, which will be described later, is 20:0.

This structure allows a probe set of the present invention to be a probe that exhibits a high detection effect in a short time under isothermal one-pot reaction conditions in a unified single step.

In the present invention, the term “probe” refers to a nucleic acid that may bind to a target nucleic acid of a complementary sequence through one or more types of chemical bonds, generally through complementary base pairing, usually through hydrogen bond formation, and thus forms a duplex structure. A probe binds or hybridizes to a “probe binding site.” In particular, a probe may be labeled with a detectable label to facilitate the detection of the probe once the probe has hybridized to a complementary target of the probe. However, alternatively, the probe may be unlabeled, but it may be detected directly or indirectly by specific binding to a labeled ligand. Probes can vary considerably in size. Generally, probes have a length of at least 7 to 15 nucleotides. Other probes may have a length of at least 20, 30, or 40 nucleotides. Still other probes are somewhat longer and have a length of at least 50, 60, 70, 80, or 90 nucleotides. Yet other probes are even longer and have a length of at least 100, 150, 200 or more nucleotides. Probes may also have any length within any range defined by any of the above values (e.g., a length of 15 to 20 nucleotides).

In the present invention, the term “molecule” refers to all types of biomolecules or cells to be detected, and biomolecules are target molecules that constitute living organisms and are responsible for their functions, the most common of which are macromolecules having a large size, such as proteins, nucleic acids, polysaccharides, and lipids, low-molecular weight substances such as organic substances including amino acids, nucleotides, monosaccharides, and vitamins, metals including iron and copper, and inorganic ions. Biomolecules extracted from the natural environment, biomolecules produced by synthesis, and biomolecules modified by attachment of initiation factors are all included. In the present invention, biomolecules may preferably be nucleic acids, proteins, and cells.

In the present invention, the term “target nucleic acid sequence” refers to all types of nucleic acids to be detected, and includes chromosomal base sequences derived from different species, subspecies, or variants, or chromosomal mutations within the same species. This includes all types of DNA, including genomic DNA, mitochondrial DNA, and viral DNA, or all types of RNA, including mRNA, ribosomal RNA, non-coding RNA, tRNA, and viral RNA, but is limited thereto.

In the present invention, the term “oligonucleotide” refers to an oligomer of nucleotides. The term “nucleic acid,” as used in the present invention, refers to a polymer of nucleotides. The term “sequence,” as used in the present invention, refers to a nucleotide sequence of an oligonucleotide or nucleic acid. The oligonucleotide or nucleic acid may be DNA, RNA, or an analog thereof (e.g., a phosphorothioate analog). The oligonucleotide or nucleic acid may also include modified bases and/or backbones (e.g., modified phosphate linkages or modified sugar moieties). Non-limiting examples of synthetic backbones that impart stability and/or other advantages to nucleic acids may include phosphorothioate linkages, peptide nucleic acids, locked nucleic acids, xylose nucleic acids, or analogs thereof.

The term “nucleic acid” includes any forms of DNA or RNA, including, for example, genomic DNA; complementary DNA (cDNA) (this is a DNA representation of messenger RNA (mRNA), usually obtained by reverse transcription or amplification of mRNA); DNA molecules produced through synthesis or amplification; and mRNA.

The term “nucleic acid” includes not only single-stranded molecules but also double- or triple-stranded nucleic acids. In a double-stranded or triple-stranded nucleic acid, the nucleic acid strands need not be coextensive (i.e., a double-stranded nucleic acid need not be double stranded along the entire length of the two strands).

The term “nucleic acid” also includes any chemical modifications thereof, such as by methylation and/or capping. Nucleic acid modifications may include the addition of chemical groups including additional charge, polarizability, hydrogen bonding, electrostatic interactions, and functionality to individual nucleic acid bases or to the nucleic acid as a whole. These modifications include sugar modifications at the C2′ position, pyrimidine modifications at the C5 position, purine modifications at the C8 position, modifications at cytosine exocyclic amines, substitution of 5-bromo-uracil, main chain modifications, and base modifications such as specific base pair combinations including isobases isocytidine and isoguanidine.

More specifically, in the probe design of the present invention, two single-stranded DNA probes are designed to expose target recognition sites so that the isothermal hybridization of a target molecule and the probe set is possible.

The “portion that binds or connects to a target molecule” included in the probe refers to, when the target molecule is a nucleic acid, a sequence that has sequence complementary to a part of a nucleic acid sequence of the target molecule and thus can hybridize with the target molecule. For example, the “portion that binds or connects to a target molecule” may have at least 90%, preferably at least 95% sequence complementary to a part of the target molecule. When the target molecule is a protein, the “portion that binds or connects to a target molecule” refers to a region that can be bind or connect to the protein that is the target molecule for an aptamer acting as a probe. The length of a “portion that binds or connects to a target molecule” may be appropriately adjusted depending on the target molecule, and is not limited to a specific length. For example, when the target molecule is miRNA, a “portion that binds or connects to a target molecule” may consist of about 22 nucleotides, but when the target molecule is mRNA or protein, a “portion that binds or connects to a target molecule” may be shorter or longer than that.

In the present invention, the term “hybridization” refers to the formation of a double-stranded nucleic acid by hydrogen bonding between single-stranded nucleic acids having complementary base sequences, and is used in a similar sense to annealing. However, in a slightly broader sense, hybridization includes cases where base sequences are completely complementary between two single strands (perfect match) as well as exceptional cases where some base sequences are not complementary (mismatch).

In the present invention, the term “complementary” refers to the ability for correct pairing between two nucleotides. In other words, when a nucleotide at a given position of a nucleic acid can form a hydrogen bond with a nucleotide of another nucleic acid, the two nucleic acids are considered complementary to each other at that position. Complementarity between two single-stranded nucleic acid molecules may be “partial,” when only some of the nucleotides are bonded, or complementarity may be complete, when total complementarity is present between single-stranded molecules. The degree of complementarity between nucleic acid strands has a considerable impact on the efficiency and strength of hybridization between nucleic acid strands.

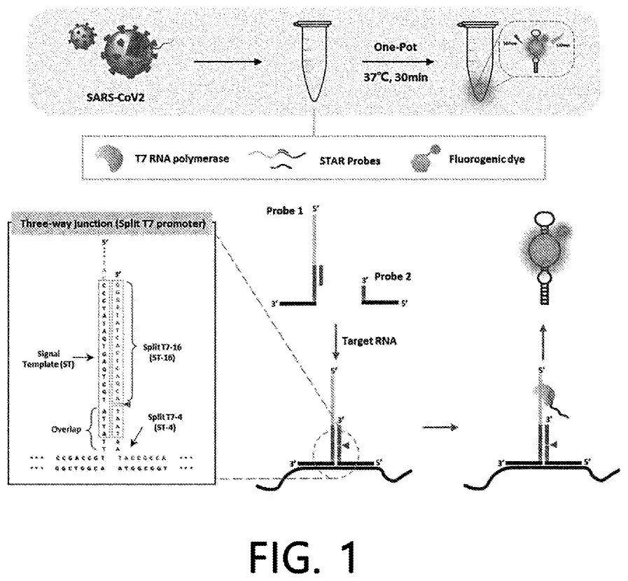

When a target molecule is a target nucleic acid sequence, the first probe binds to an upstream hybridization sequence (UHS) of the target nucleic acid sequence, has a sequence complementary to a T7 promoter, and has an aptamer sequence. Ya included in General Formula II of the first probe corresponds to a partial sequence of the T7 promoter, and Yb in General Formula III of the second probe also corresponds to the remaining partial sequence of the T7 promoter, and Ya and Yb are contiguous sequences constituting one T7 promoter and correspond to two split fragments. In the presence of target RNA, the probe set forms a three-way junction structure, resulting in a double-stranded T7 promoter having a nick site. Therefore, in the absence of a target nucleic acid sequence, a T7 promoter sequence is split into Ya and Yb, so a polymerase may not bind thereto, and only in the presence of a target nucleic acid sequence, a polymerase may bind thereto to initiate the transcription of the aptamer portion of the first probe (see FIG. 1).

Since a signal is generated in response to a specific substance by the aptamer sequence region X of the first probe, the final product may be identified.

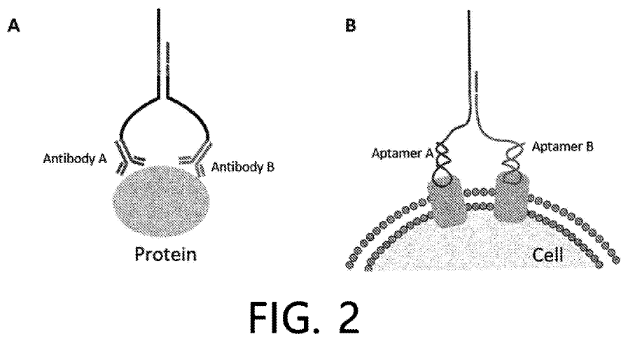

When a target molecule is a protein or cell, the first probe and the second probe each have a portion that binds or connects to the target protein or cell. For example, as shown in FIG. 2, an antibody or aptamer that specifically binds to a target site of a protein or cell may be present in a bound or coupled form. An aptamer may be coupled through a complementary sequence such as oligonucleotides (DNA, etc.) of a certain length (˜several tens of nucleotides), and an antibody may be coupled through N-hydroxysuccinimide (NHS) ester synthesis or the like.

As in the case where the target nucleic acid sequence is present, in the presence of a target protein or cell, the probe set forms a three-way junction structure, and a double-stranded T7 promoter having a nick site is completed. Therefore, when the target protein is not present, the T7 promoter sequence is split into Ya and Yb, so a polymerase may not bind, and only when the target protein or cell is present, a polymerase binds, and the transcription of the aptamer region of a first probe is initiated (see FIG. 2).

In the present invention, an “aptamer” used as a reporter is a single-stranded nucleic acid (DNA, RNA or modified nucleic acid) that has a stable tertiary structure in itself and is able to bind to a target with high affinity and specificity.

Since the development of an aptamer discovery technology called Systematic Evolution of Ligands by EXponential enrichment (SELEX), aptamers capable of binding to various target molecules, including low-molecular weight organic substances, peptides, and membrane proteins, have been continuously discovered. Aptamers are often compared to single antibodies due to their inherent high affinity (usually at a pM level) and the ability to specifically bind to a target molecule, and in particular, they have high potential as alternative antibodies to the extent that they are called “chemical antibodies.” In addition, the binding of an aptamer to a specific chemical molecule may be confirmed by methods such as fluorescence emission, because the absorption and emission wavelength bands are different depending on the binding substances.

As an aptamer of the present invention and a reactive material interacting with the aptamer, any type of aptamer and reactive material may be used, as long as they may generate a detectable signal as a desired effect.

According to a preferred embodiment of the present invention, when a target molecule is a target nucleic acid sequence, when the first probe and the second probe hybridize with a target nucleic acid sequence, the first probe and the second probe are located at positions immediately adjacent to each other.

One T7 promoter structure is formed when Y of the first probe; and Ya of the first probe and Yb of the second probe are hybridized. The term “adjacent,” as used in the present specification when referring to a hybridization position of the first probe and the second probe, means that the ends of Ya and Yb in the two probes are positioned so that they may be connected to each other, and Ya and Yb are sufficiently close so that they may hybridize with Y.

In one embodiment of the present invention, a target molecule may be a protein, peptide, nucleic acid, carbohydrate, lipid, polysaccharide, glycoprotein, hormone, receptor, antigen, antibody, virus, pathogen, toxic substance, substrate, metabolite, ATP, cocaine, mercury, transition state analog, cofactor, inhibitor, drug, dye, nutrient, growth factor, cell, tissue, or the like, but is not limited thereto, and it may be almost any chemical or biological effector, and it refers to available target molecules of any sizes. According to a preferred embodiment of the present invention, the target molecule may be a nucleic acid, protein, cell, and ATP.

According to a preferred embodiment of the present invention, when the target molecule is a nucleic acid, Z and Z′ may be nucleic acid sequences that complementarily bind to the nucleic acid to be detected, and when the target molecule is a protein, Z and Z′ may be antibodies or aptamers capable of binding to the protein, and when the target molecule is a cell, Z and Z′ may be antibodies or aptamers capable of binding to any one selected from the group consisting of extracellular proteins, cellular phospholipids, bacterial peptidoglycans, and LPS, and when the target molecule is ATP, Z and Z′ may be split aptamers capable of binding to ATP.

Specifically, when the target molecule is ATP, preferably, since a substance such as ATP corresponds to a small molecule, Z and Z′ may be split aptamer portions capable of binding to ATP. In detail, refer to the content disclosed in the paper, A. Chen et al. “Split aptamers and their applications in sandwich aptasensors,” Trends in Analytical Chemistry, 80 (2016), pp. 581-593. As described therein, when a target molecule is ATP, a DNA aptamer that binds to intact ATP may be cut into halves to produce two strands of DNA aptamers (split DNA aptamers) and used according to a method known in the art.

Representative portions that bind or connect to a target molecule include antibodies, peptides, nucleic acids, and aptamers.

An antibody is a substance that specifically binds to an epitope of an antigen and causes an antigen-antibody reaction. An aptamer refers to a small (20 to 60 nucleotides) fragment of a single-stranded nucleic acid (DNA or RNA) capable of specifically binding to various types of substances, from low-molecular weight compounds to proteins, with high affinity.

The probe may further include a linker portion bound between an aptamer sequence portion having an interactive labeling system including one label or multiple labels generating the detectable signal and a portion bound or linked to the target molecule. In the present invention, the linker portion may be introduced at one end of a probe to reach a portion that binds or connects to a target molecule, and the specific type of the linker portion is not particularly limited, but for example, the linker portion may be a short nucleic acid sequence of 4 to 10 nucleotides.

According to a preferred embodiment of the present invention, in the probe set of the present invention, the Ya and Yb may be partial sequences of the T7 promoter, and when Ya and Yb are sequentially arranged in the order of Ya and the Yb, they may form the T7 promoter, and a Ya:Yb split ratio may be 20:0 to 15:5.

Since the T7 promoter sequence is split into the first probe and the second probe, when a target molecule is not present, a polymerase may not bind to the T7 promoter, and transcription may not be initiated, and ultimately, no signal is generated from the label on the transcription products of the first probe and the second probe, and thus detection of the target molecule is not achieved. When a target molecule is present, Ya of the first probe and Yb of the second probe are sequentially arranged to form a T7 promoter, and a transcription process is initiated by a polymerase. When the transcription process is initiated, a product of X of the first probe containing the aptamer sequence may be produced.

In the existing signal mediated amplification of RNA technology (SMART), a DNA polymerase was used and then an RNA polymerase was sequentially used, so two enzymes had to be used. Accordingly, because a non-specific signal is generated when two enzymes are present together, a one-step reaction was impossible, and the reaction also took a long time. However, the present invention omitted a step of generating a T7 RNA promoter using a DNA polymerase, and instead, the T7 promoter was split to prevent the T7 promoter itself from being generated when a target is not present. Through this, a system capable of detecting a target molecule with only one polymerase was introduced, and since only one enzyme is used, the generation of non-specific signals is reduced, and since the reaction time is shortened, rapid reaction is possible.

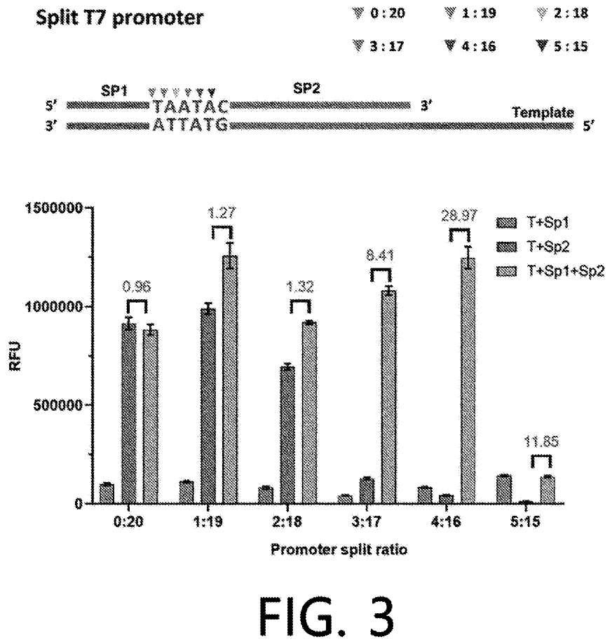

In other words, since the core of the present invention is the effective formation of a split T7 promoter in the presence of a target molecule by inducing a three-way junction structure, in the present invention, an existing T7 promoter was split, and the transcription efficiency according to each ratio was analyzed. Specifically, in one embodiment of the present invention, in Example 1-1, in order to control the formation of a T7 promoter, nick sites were sequentially inserted starting from the 5′ end of the T7 promoter to prepare T7 promoters split at various ratios. A complementary sequence of an aptamer was arranged on a template and transcribed, and a transcription reaction of the resulting product was monitored. As shown in FIG. 3, as a result of sequentially inserting from 0:20 to 5:15 starting from the 5′ end region, the fluorescence value rapidly decreased after a ratio of 3:17 due to the absence of SPi, which indicated that the 3-nucleotide portion of the 5′ side is important for the T7 RNA polymerase to specifically recognize the T7 promoter. It was confirmed that at 5:15, transcription hardly progressed even when both SP1 and SP2 were present, indicating that when a nick site is present after a 5-mer on the 5′ side of the T7 promoter, the T7 RNA polymerase may not recognize the T7 promoter normally. It was confirmed that in order to form a complete double-stranded T7 promoter through the formation of a three-way junction structure, a split ratio of 4:16 from the 5′ end region is most suitable.

Therefore, in one embodiment of the present invention, a split ratio may be appropriately adjusted, and most preferably, a Ya:Yb split ratio may be 16:4.

In the probe set of the present invention, the label may be selected from the group consisting of a chemical label, an enzymatic label, a radioactive label, a fluorescent label, a luminescent label, a chemiluminescent label, and a metallic label.

In one preferred embodiment of the present invention, General Formula I of the first probe may be modified into General Formula I′ further including an overlapping sequence W between Y and Z;

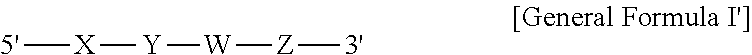

and General Formula III of the second probe may be modified into General Formula III′, further including an overlapping sequence W′ between Yb and Z′;

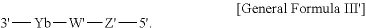

The overlapping sequence was introduced to overcome the problem that when a target molecule is present, a three-way junction structure is not completely formed due to excessively short overlapping, so the T7 promoter is not formed and transcription is not carried out effectively. The overlapping sequences W and W′ are complementary to each other.

Therefore, in a preferred embodiment, specifically, in Example 1-2, structural optimization was performed to apply the T7 promoter with a Ya:Yb ratio of 16:4 to the three-way junction structure. The 16 nucleotides on the 3′ side of the T7 promoter bind complementarily to a single template (Signal template, ST) and are universal. Only when a target RNA is present, since a first probe consisting of ST and split T7-16 (Split T7-16, ST-16) and a second probe (Split T7-4, ST-4) need to be combined to form a complete T7 promoter with a nick site, a Tm value of an overlapping sequence portion is the most important factor, and the Tm value was adjusted by arranging an additional overlapping sequence (0 to 4 bp).

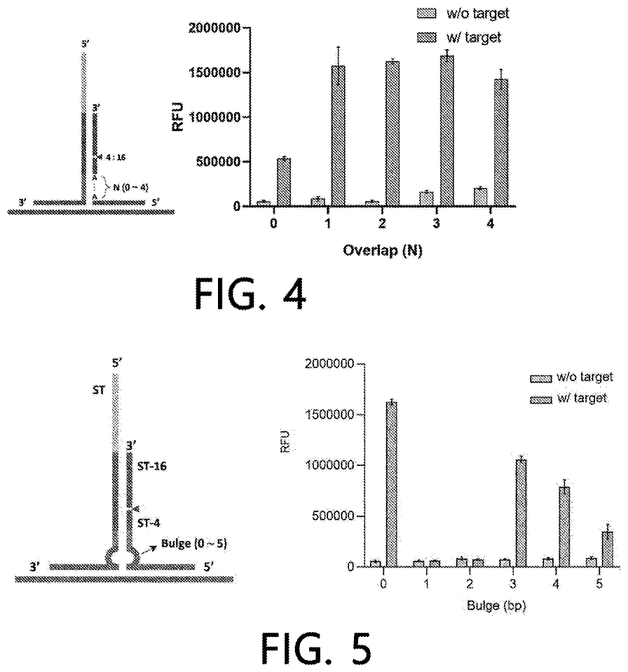

As a result, as shown in FIG. 4, it was confirmed that when an overlapping sequence (N=0) is 4 bp of a T7 promoter, no fluorescence is produced when a target sequence is not present, so it is suitable for the purpose of not forming a structure, but when a target nucleic acid sequence is present, a three-way junction structure is not completely formed due to excessively short overlapping, so the T7 promoter is not formed, and transcription is not performed effectively. It was confirmed that when an overlapping sequence of 1 bp or more was added (N=1), a high fluorescence value was observed only when a target sequence was present. Through this, it was confirmed that in order to stably form a three-way junction structure and induce a transcriptional reaction, an overlap of at least 1 bp is required in addition to 4 bp from the T7 promoter. When an added overlapping sequence exceeded 3 bp, non-specific transcription occurred even when no target sequence was present, so the appropriate additional overlapping sequence was set to 2 bp, and the total overlapping portion was set to 6 bp (4 bp from the T7 promoter). In addition, as confirmed in Example 1-2, when they are not complementary, a bulge may be formed in the three-way junction structure, which may reduce the stability of the structure (FIG. 5).

In the probe set of the present invention, the length of the overlapping sequences W and W′ may be 1 bp or 2 bp.

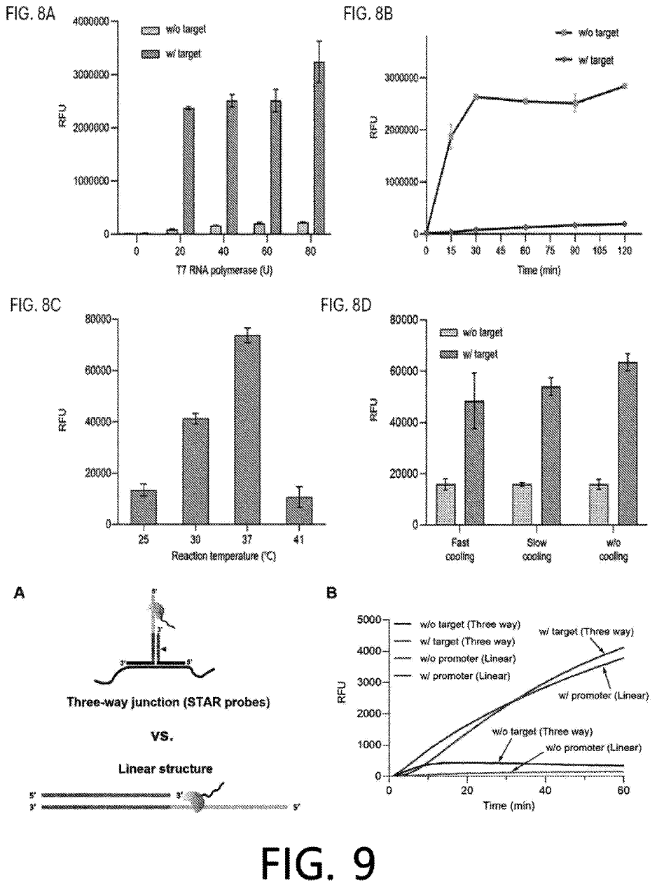

In a probe set of the present invention, the isothermal one-pot reaction may be performed at a constant temperature in a range of 15° C. to 50° C. The temperature in the range of 15° C. to 50° C. is a temperature known in the art at which enzymes act, and the temperature is not limited thereto as long as the desired effect of the present invention may be obtained. However, as confirmed in Example 1-3 of the present invention, it was confirmed that the efficiency of the reaction of the present invention was the best at 37° C. (FIG. 8C). Therefore, the constant temperature of the isothermal one-pot reaction is preferably 34° C. to 40° C.

In the probe set of the present invention, the probe set may allow circular RNA to be detected separately from linear RNA. Specifically, as shown in FIG. 12, the first probe and the second probe of the present invention may each bind to both ends of linear RNA, and the first probe and the second probe may bind to each other only when both ends of linear RNA bind to generate circular RNA. Therefore, using the probe set of the present invention, a T7 promoter may be formed and transcription may occur only in the case of circular RNA, unlike linear RNA, so the probe set of the present invention may be used to detect circular RNA.

In the probe set of the present invention, the polymerase may be selected from the group consisting of bacteriophage T7 RNA polymerase, bacteriophage T3 polymerase, bacteriophage RNA polymerase, bacteriophage (DII polymerase, Salmonella bacteriophage sp6 polymerase, Pseudomonas bacteriophage gh-1 polymerase, E. coli RNA polymerase holoenzyme, E. coli RNA polymerase core enzyme, human RNA polymerase I, human RNA polymerase II, human RNA polymerase III, human mitochondrial RNA polymerase, and variants thereof, but is not limited thereto.

In one embodiment of the present invention, bacteriophage T7 RNA polymerase was used as a polymerase.

In the probe set of the present invention, the isothermal one-pot reaction may be performed in a unified and simultaneous manner with a one-pot reaction solution containing Tris-HCl, MgCl₂, dithiothreitol (DTT), spermidine, ribonucleotide triphosphates (rNTPs), an RNase inhibitor, and a single-stranded DNA binding protein (SSB).

Specifically, in one embodiment, in Example 1-3, an optimization experiment was performed on a buffer, enzyme, reaction time, and reaction temperature to obtain the best performance of the proposed probe set.

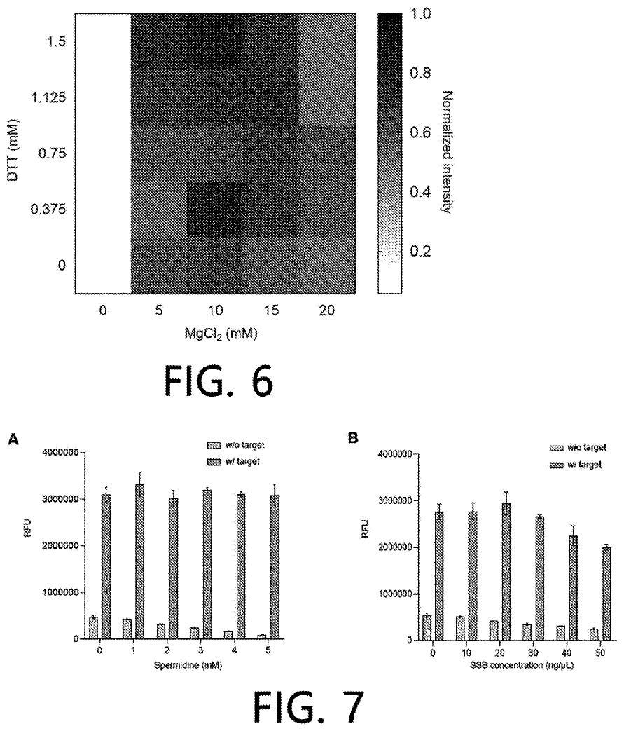

In the present invention, the one-pot reaction solution may preferably include 1 to 50 mM of MgCl₂, 0.1 to 10 mM of DTT, 0.1 to 10 mM of spermidine, 1 to 100 ng/μL of SSB, and 1 to 100 U of T7 RNA polymerase, more preferably 1 to 30 of mM MgCl₂, 0.5 to 5 mM of DTT, 1 to 5 mM of spermidine, 10 to 50 ng/μL of SSB, and 10 to 50 U of T7 RNA polymerase, most preferably 10 mM of MgCl₂, 1.5 mM of DTT, 3 mM of spermidine, 20 ng/μL of SSB, and 20 U of T7 RNA polymerase (FIGS. 6 to 9).

The present invention also provides a composition for detecting a target molecule, including the isothermal one-pot reaction probe set for detecting a target molecule.

The composition for detecting a target molecule may further include tools and/or reagents commonly used for the detection and/or analysis of a target molecule, in addition to nucleic acid sequences and peptides that specifically bind to the target molecule. These tools or reagents may include suitable carriers, labels capable of generating a detectable signal, solubilizers, detergents, buffers, stabilizers, and the like. When a labeling substance is a fluorescent substance, a substrate needed to measure fluorescence intensity may further be included. Suitable carriers may include soluble carriers, for example, physiologically acceptable buffers known in the art (e.g., PBS, etc.); insoluble carriers, for example, polystyrene, polyethylene, polypropylene, polyester, polyacrylonitrile, fluororesins, cross-linked dextran, polysaccharides, and the like; polymers such as magnetic particles plated with a metal on latex, and paper, glass, metal, agarose, and/or combinations thereof.

In the composition of the present invention, the composition may include two or more types of isothermal one-pot reaction probe sets for detecting two or more types of target molecules.

In the composition of the present invention, the two or more types of isothermal one-pot reaction probe set for detecting target molecules may each include different interactive labeling systems, and the two or more types of probe sets may respectively bind to different target molecules to enable multiplex detection of different target molecules.

In other words, the two or more types of targets may be different target sites present in a single pathogen, and in this case, more accurate and precise diagnosis is possible by effectively detecting different sites of the target.

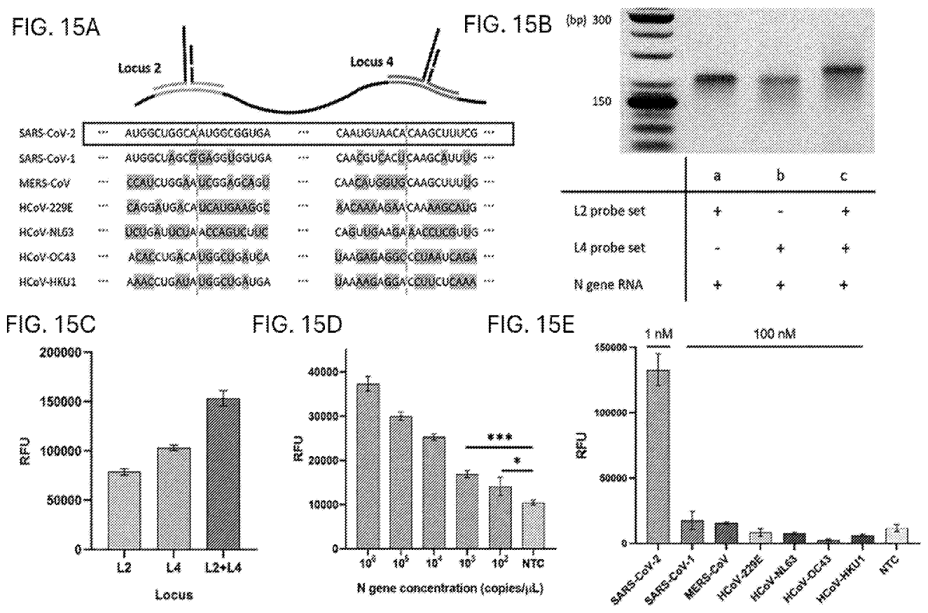

Specifically, in Examples 2 and 3 of the present invention, probes for detection were designed to target different target sites using SARS-CoV-2 as a target substance. As a result, probes targeting two genetic loci indicated as L2 and L4 were designed, and the results showed that the two probes specifically bind to each region of the target nucleic acid sequence (see FIGS. 15A-15E). Therefore, more accurate detection is possible because different parts of the target are effectively detected.

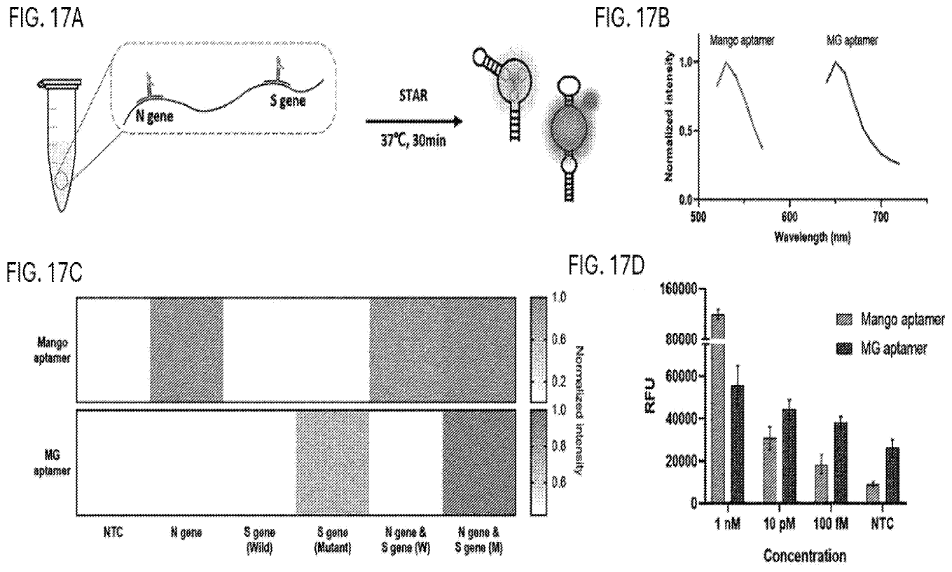

Furthermore, simultaneous detection of a D614G mutation and an N gene of SARS-CoV-2 in one tube was tested, in which a mango aptamer and a malachite green aptamer allowed the N gene and D614G of SARS-CoV-2 to emit fluorescence without interference, respectively (FIG. 17C). It was confirmed that targets for the N gene and D614G could be analyzed simultaneously up to 100 fM, so that highly sensitive detection is possible (FIG. 17D).

In addition, the two or more types of targets may be interactively different molecular diagnostic targets, for example, infectious harmful microorganisms, and in this case, simultaneous detection and diagnosis of different pathogens are possible.

Specifically, in Example 6 of the present invention, detection of two pathogenic bacteria was performed, and as a result, it was confirmed that both E. coli and Methicillin-resistant Staphylococcus aureus (MRSA) specifically bound to each probe and generated high fluorescence signals. Therefore, the present invention allows detection and diagnosis of different pathogens individually or simultaneously.

In another preferred embodiment of the present invention, the isothermal one-pot reaction may be performed at a constant temperature in a range of 15° C. to 50° C.

The present invention also provides a kit for detecting a target molecule, including the composition for detecting a target molecule, a polymerase, and an isothermal one-pot reaction solution.

The kit of the present invention described above in the present specification may further include various polynucleotide molecules and enzymes, and various buffers and reagents. In addition, the kit of the present invention may include reagents necessary for carrying out positive control and negative control reactions. An optimal amount of reagent to be used in any one particular reaction may be easily determined by one skilled in the art upon learning the disclosure in the present specification.

Since the kit of the present invention is manufactured to perform target molecule detection using the probe set of the present invention described above, description of duplicate content is omitted to avoid complexity of the present specification.

In addition, the present invention provides a method of detecting a target molecule, using the isothermal one-pot reaction probe set for detecting a target molecule, and specifically, the method may include the following steps:
(a) treating a sample with an isothermal one-pot reaction probe set for detecting a target molecule consisting of a first probe and a second probe of the present invention to bind to or hybridize with a target molecule;
(b) treating a hybridization product of Step (a) with a polymerase to initiate transcription; and
(c) treating a transcription product of Step (b) with an aptamer-reactive substance to detect signal generation of an aptamer in the transcription product.

At this time, the signal generation in Step (c) is characterized in that it indicates the presence of a target molecule in the sample.

The method of detecting a target molecule of the present invention is characterized in that a target molecule is detected under isothermal single reaction conditions without a separate amplification reaction.

Therefore, the method of detecting a target molecule using the probe set of the present invention is designed so that a hybridization step, a transcription reaction, and an aptamer signal reaction occur simultaneously, and Steps (a) to (c) of the present invention may be simultaneously performed in one container.

In addition, an aptamer used to confirm a final signal is not limited to a malachite green aptamer, and any type of RNA-based fluorescent aptamer may be used.

In one embodiment of the present invention, an isothermal nucleic acid amplification reaction may be performed prior to the method of detecting a target molecule using the isothermal one-pot reaction probe set for detecting a target molecule.

The type of isothermal nucleic acid amplification reaction is not particularly limited, but in one embodiment of the present invention, the isothermal nucleic acid amplification reaction may be recombinase polymerase amplification (RPA).

The term “recombinant-polymerase amplification method,” as used in the present specification, refers to a technology of confirming DNA and RNA amplification, and unlike conventional PCR, it is a technology using a DNA-binding protein and a recombinase to quickly and accurately amplify a target sequence. Since an RPA method allows amplification even under isothermal conditions using a mesophilic polymerase, it has the advantage that viruses may be detected and diagnosed in a short period of time using an isothermal device without special equipment.

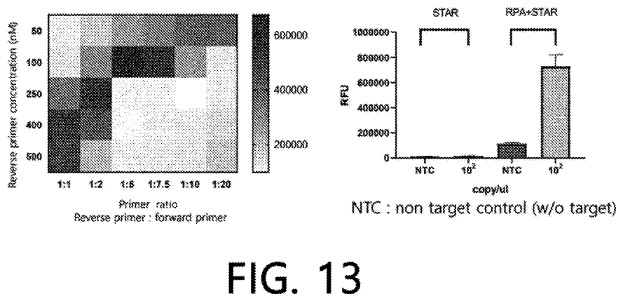

It was confirmed that when RPA was performed before Step (a), the signal was amplified compared to when the STAR reaction of the present invention was performed (see FIG. 13). Therefore, in connection with the present invention, an isothermal nucleic acid amplification reaction may be performed as a method of detecting a target molecule more sensitively.

The present invention also provides an on-site molecular diagnostic method using the isothermal one-pot reaction probe set for detecting a target molecule.

In the specification of the present invention, “on-site molecular diagnostic method” refers to a method that allows on-site diagnosis in medical settings and precisely diagnoses a disease by directly testing a molecular level within cells and genes (RNA, DNA) of a pathogen.

The on-site molecular diagnostic method may include the following steps:
(a) treating a sample with an isothermal one-pot reaction probe set for detecting a target molecule consisting of a first probe and a second probe to hybridize with a target molecule;
(b) treating a hybridization product of Step (a) with a polymerase to initiate transcription; and
(c) treating a transcription product of Step (b) with an aptamer-reactive substance to detect signal generation of an aptamer in the transcription product.

At this time, the signal generation in Step (c) is characterized in that it indicates the presence of a target molecule in the sample.

In the on-site molecular diagnostic method of the present invention, the molecular diagnostic method may be used to detect a pathogenic microorganism.

In the specification of the present invention, “pathogenic microorganism” refers to a microorganism that parasitizes the human body and causes a disease, is not limited to the pathogenic microorganisms described in the embodiments of the present invention, and all pathogenic microorganisms may be applied.

In the on-site molecular diagnostic method of the present invention, the pathogenic microorganisms may be one or more selected from the group consisting of Staphylococcus Aureus, Vibrio vulnificus, E. coli, Middle East respiratory syndrome coronavirus, influenza A virus, severe acute respiratory syndrome coronavirus, respiratory syncytial virus (RSV), human immunodeficiency virus (HIV), herpes simplex virus (HSV), human papilloma virus (HPV), human parainfluenza viruses (HPIV), dengue virus, hepatitis B virus (HBV), yellow fever virus, rabies virus, Plasmodium, cytomegalovirus (CMV), Mycobacterium tuberculosis, Chlamydia trachomatis, rotavirus, human metapneumovirus (hMPV), Crimean-Congo hemorrhagic fever virus, Ebola virus, Zika virus, henipavirus, norovirus, Lassa virus, rhinovirus, flavivirus, Rift Valley fever virus, hand-foot-mouth disease virus, Salmonella sp., Shigella sp., Enterobacteriaceae sp., Pseudomonas sp., Moraxella sp., Helicobacter sp., and Stenotrophomonas sp.

The present invention also provides an isothermal one-pot reaction probe set for detecting SARS-CoV-2 and/or detecting a mutant thereof, including a third probe and a fourth probe,
wherein the third probe is a PP having a structure including General Formulas IV and V below,

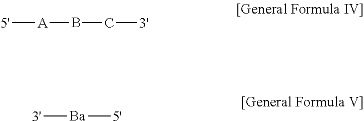

wherein in General Formula IV, A is an aptamer sequence portion having an interactive labeling system including one label or multiple labels generating a detectable signal;
B is a sequence complementary to a T7 promoter;
C is an upstream hybridization sequence (UHS) portion having a hybridization sequence complementary to a nucleic acid sequence of SARS-CoV-2 and/or a mutant thereof; the nucleic acid sequence of SARS-CoV-2 and/or a mutant thereof is DNA or RNA; and
A, B and C are deoxyribonucleotides; and
in General Formula V, Ba is a partial sequence of a T7 promoter; and Ba is a deoxyribonucleotide;
wherein the fourth probe is a PP having a structure consisting of General Formula VI,

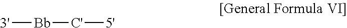

wherein in General Formula VI, Bb is absent or a partial sequence of the T7 promoter;
C′ is a downstream hybridization sequence (DHS) portion having a hybridization sequence complementary to a nucleic acid sequence of SARS-CoV-2 and/or a mutant thereof; the nucleic acid sequence of SARS-CoV-2 and/or a mutant thereof is DNA or RNA; and
Bb and C′ are deoxyribonucleotides;
wherein the third probe and the fourth probe are hybridized with a nucleic acid sequence of SARS-CoV-2 and/or a mutant thereof and then transcription is initiated by a polymerase to generate a signal.

The third probe of the probe set of the present invention includes a structure including the following three different portions within one oligonucleotide molecule, and another oligonucleotide molecule: A, which is an aptamer sequence portion having an interactive labeling system including one label or multiple labels generating a detectable signal; B, which is a sequence complementary to a T7 promoter; and C, which is a portion that is hybridized with SARS-CoV-2 and/or a mutant thereof; and Ba is a partial sequence of the T7 promoter complementary to B.

In addition, the fourth probe of the probe set of the present invention also has a structure including one or two unique different portions within one oligonucleotide molecule: Bb, which is complementary to B and a partial sequence of the T7 promoter and has a contiguous sequence with Ba; and C′, which is a portion that is hybridized with SARS-CoV-2 and/or a mutant thereof. Bb may not be present when a split ratio, which will be described later, is 20:0.

This structure allows the probe set of the present invention to be a probe that exhibits a high detection effect in a short time under isothermal one-pot reaction conditions in a unified single step.

More specifically, the probe of the present invention is designed so that two single-stranded DNA probes expose target recognition portions, enabling isothermal hybridization of SARS-CoV-2 and the probe set.

C of the third probe binds to a UHS of SARS-CoV-2 and/or a mutant thereof, has a sequence complementary to a T7 promoter, and has an aptamer sequence. Ba included in General Formula V of the third probe corresponds to a partial sequence of the T7 promoter, and Bb in General Formula VI of the fourth probe also corresponds to the remaining partial sequence of the T7 promoter, and Ba and Bb are contiguous sequences constituting one T7 promoter and correspond to two split fragments. In the presence of SARS-CoV-2 and/or a mutant thereof, the probe set forms a three-way junction structure, resulting in a double-stranded T7 promoter having a nick site. Therefore, in the absence of SARS-CoV-2 and/or a mutant thereof, a T7 promoter sequence is split into Ba and Bb, so a polymerase may not bind thereto, and only in the presence of SARS-CoV-2 and/or a mutant thereof, a polymerase may bind thereto to initiate the transcription of the aptamer portion of the third probe (see FIG. 1).

Since a signal is generated in response to a specific substance by the aptamer sequence region A of the third probe, the final product may be identified.

According to a preferred embodiment of the present invention, when the third probe and the fourth probe hybridize with SARS-CoV-2 and/or a mutant thereof, the third probe and the fourth probe are located at positions immediately adjacent to each other.

One T7 promoter structure is formed when B of the third probe; and Ba of the third probe and Bb of the fourth probe are hybridized. The term “adjacent,” as used in the present specification when referring to a hybridization position of a third probe and a fourth probe, means that the ends of Ba and Bb in the two probes are positioned so that they may be connected to each other, and Ba and Bb are sufficiently close so that they may hybridize with B.

The probe may further include a linker portion bound between an aptamer sequence portion having an interactive labeling system including one label or multiple labels generating the detectable signal and a portion bound or linked to the target molecule. In the present invention, the linker portion may be introduced at one end of a probe to reach a portion that binds or connects to a target molecule, and the specific type of the linker portion is not particularly limited, but for example, the linker portion may be a short nucleic acid sequence of 4 to 10 nucleotides.

According to one preferred embodiment of the present invention, in the probe set of the present invention, the Ba and Bb may be partial sequences of a T7 promoter, and when Ba and Bb are sequentially arranged in the order of Ba and Bb, they may form the T7 promoter, and a Ba:Bb split ratio may be 20:0 to 15:5.

Since the T7 promoter sequence is split into the third probe and the fourth probe, when SARS-CoV-2 and/or a mutant thereof is not present, a polymerase may not bind to the T7 promoter, and transcription may not be initiated, and ultimately, no signal is generated from the label on the transcription products of the third probe and the fourth probe, and thus detection of the target molecule is not achieved. When SARS-CoV-2 and/or a mutant thereof is present, Ba of the third probe and Bb of the fourth probe are sequentially arranged to form a T7 promoter, and a transcription process is initiated by a polymerase. When the transcription process is initiated, a product of A of the third probe containing the aptamer sequence may be produced.

In one embodiment of the present invention, a split ratio may be appropriately adjusted, and most preferably, a Ba:Bb split ratio may be 16:4.

In a probe set of the present invention, the label may be selected from the group consisting of a chemical label, an enzymatic label, a radioactive label, a fluorescent label, a luminescent label, a chemiluminescent label, and a metallic label.

In one preferred embodiment of the present invention, an isothermal one-pot reaction probe set for detecting SARS-CoV-2 and/or detecting a mutant thereof may be provided, wherein General Formula IV of the third probe may be modified into General Formula IV′ further including an overlapping sequence D between B and C;

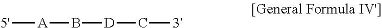

and General Formula VI of the fourth probe may be modified into General Formula VI′ further including an overlapping sequence D′ between Bb and C′;

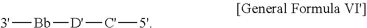

The overlapping sequence was introduced to overcome the problem that when SARS-CoV-2 and/or a mutant thereof is present, a three-way junction structure is not completely formed due to excessively short overlapping, so the T7 promoter is not formed and transcription is not carried out effectively. The overlapping sequences D and D′ are complementary to each other.

Therefore, in a preferred embodiment, specifically, in Example 1-2, structural optimization was performed to apply the T7 promoter with a Ba:Bb ratio of 16:4 to the three-way junction structure. The 16 nucleotides on the 3′ side of the T7 promoter bind complementarily to a single template (signal template, ST) and are universal. Only when a target RNA is present, since a first probe consisting of ST and split T7-16 (Split T7-16, ST-16) and a second probe (Split T7-4, ST-4) need to be combined to form a complete T7 promoter with a nick site, a Tm value of an overlapping sequence portion is the most important factor, and the Tm value was adjusted by arranging an additional overlapping sequence (0 to 4 bp).

As a result, as shown in FIG. 4, it was confirmed that when an overlapping sequence (N=0) is 4 bp of a T7 promoter, no fluorescence is produced when SARS-CoV-2 is not present, so it is suitable for the purpose of not forming a structure, but when SARS-CoV-2 is present, a three-way junction structure is not completely formed due to excessively short overlapping, so the T7 promoter is not formed, and transcription is not performed effectively. It was confirmed that when an overlapping sequence of 1 bp or more was added (N=1), a high fluorescence value was observed only when SARS-CoV-2 was present. Through this, it was confirmed that in order to stably form a three-way junction structure and induce a transcriptional reaction, an overlap of at least 1 bp is required in addition to 4 bp from the T7 promoter. When an added overlapping sequence exceeded 3 bp, non-specific transcription occurred even when no SARS-CoV-2 was present, so the appropriate additional overlapping sequence was set to 2 bp, and the total overlapping portion was set to 6 bp (4 bp from the T7 promoter). In addition, as confirmed in Example 1-2, when they are not complementary, a bulge may be formed in the three-way junction structure, which may reduce the stability of the structure (FIG. 5).

In one embodiment of the present invention, the length of the overlapping sequences D and D′ may be 1 bp or 2 bp.

In the isothermal one-pot reaction probe set for detecting SARS-CoV-2 and/or detecting a mutant thereof of the present invention, the isothermal one-pot reaction may be performed at a constant temperature in a range of 15° C. to 50° C. The temperature in the range of 15° C. to 50° C. is a temperature known in the art at which enzymes act, and the temperature is not limited thereto as long as the desired effect of the present invention may be obtained. However, as confirmed in Example 1-3 of the present invention, it was confirmed that the efficiency of the reaction of the present invention was the best at 37° C. (FIG. 8C). Therefore, the constant temperature of the isothermal one-pot reaction is preferably 34° C. to 40° C.

In the isothermal one-pot reaction probe set for detecting SARS-CoV-2 and/or detecting a mutant thereof of the present invention, the polymerase may be selected from the group consisting of bacteriophage T7 RNA polymerase, bacteriophage T3 polymerase, bacteriophage RNA polymerase, bacteriophage (DII polymerase, Salmonella bacteriophage sp6 polymerase, Pseudomonas bacteriophage gh-1 polymerase, E. coli RNA polymerase holoenzyme, E. coli RNA polymerase core enzyme, human RNA polymerase I, human RNA polymerase II, human RNA polymerase III, human mitochondrial RNA polymerase, and variants thereof, but is not limited thereto. In one embodiment of the present invention, bacteriophage T7 RNA polymerase was used as a polymerase.

In the isothermal one-pot reaction probe set for detecting SARS-CoV-2 and/or detecting a mutant thereof of the present invention, the isothermal one-pot reaction may be performed in a unified and simultaneous manner with a one-pot reaction solution containing Tris-HCl, MgCl₂, DTT, spermidine, rNTPs, an RNase inhibitor, and an SSB.

Specifically, in one embodiment, in Example 1-3, an optimization experiment was performed on a buffer, enzyme, reaction time, and reaction temperature to obtain the best performance of the proposed probe set.

In the present invention, the one-pot reaction solution may preferably include 1 to 50 mM of MgCl₂, 0.1 to 10 mM of DTT, 0.1 to 10 mM of spermidine, 1 to 100 ng/μL of SSB, and 1 to 100 U of T7 RNA polymerase, more preferably 1 to 30 of mM MgCl₂, 0.5 to 5 mM of DTT, 1 to 5 mM of spermidine, 10 to 50 ng/μL of SSB, and 10 to 50 U of T7 RNA polymerase, most preferably 10 mM of MgCl₂, 1.5 mM of DTT, 3 mM of spermidine, 20 ng/μL of SSB, and 20 U of T7 RNA polymerase (FIGS. 6 to 9).

In the probe set of the present invention, a region of the nucleic acid sequence of SARS-CoV-2 and/or a mutant thereof is an N gene or an S gene

In the probe set of the present invention, the isothermal one-pot reaction probe set for detecting SARS-CoV-2 may be specifically bind to an N gene and may include at least one probe set selected from the group consisting of a probe set in which the third probe is SEQ ID NO: 37 and the fourth probe is SEQ ID NO: 38 and a probe set in which the third probe is SEQ ID NO: 41 and the fourth probe is SEQ ID NO: 42.

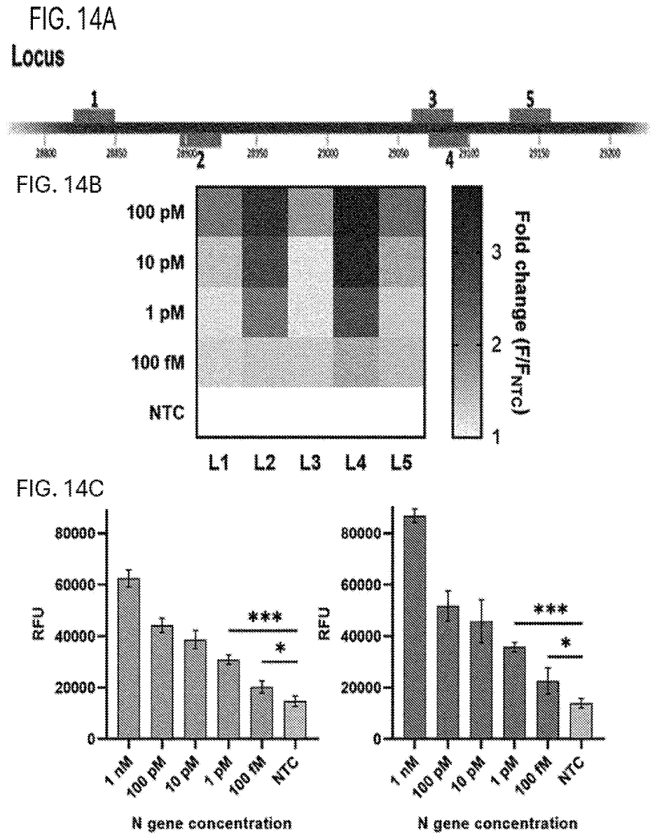

Specifically, in Examples 2 and 3 of the present invention, probes for detection were designed to target different regions within an N gene using the N gene of SARS-CoV-2 as a target material. As a result, probes targeting two genetic loci indicated as L2 and L4 were designed, and it was found that the two probes specifically bind to each region of the target nucleic acid sequence (see FIGS. 14A-14C). The third probe binding to L2 is a probe consisting of SEQ ID NO: 37, and the fourth probe is a probe consisting of SEQ ID NO: 38. The third probe binding to L4 is a probe consisting of SEQ ID NO: 41, and the fourth probe is a probe consisting of SEQ ID NO: 42. When each of these is used, quantitative detection up to 100 fM is possible (FIG. 14C).

Furthermore, it was confirmed that when probes binding to L2 and L4 were treated simultaneously, the fluorescence intensity increased compared to a case where a single probe was used (FIG. 15C). Therefore, the method may be used as a method of improving signals in limited situations where the RNA concentration of targeted SARS-CoV-2 or a mutant thereof is low.

Meanwhile, the G-type virus, in which amino acid 614 of the spike protein, which plays an important role in the virus's invasion into cells, is changed from aspartic acid (D) to glycine (G), has drastically increased in Europe and the United States since March, and it is currently found in most regions. According to recent reports, it was confirmed that more than 70 coronavirus variants have been generated, and there are 8 variants with increased transmissibility (D614G, etc.), including the S type (the amino acid D at position 614 of the S protein) and the G type (the amino acid G at position 614 of the S protein).

In the present invention, detection was performed for the D614G mutation, but is not limited thereto. When a target is determined, various mutations may be detected by adjusting the probe sequence through programming.

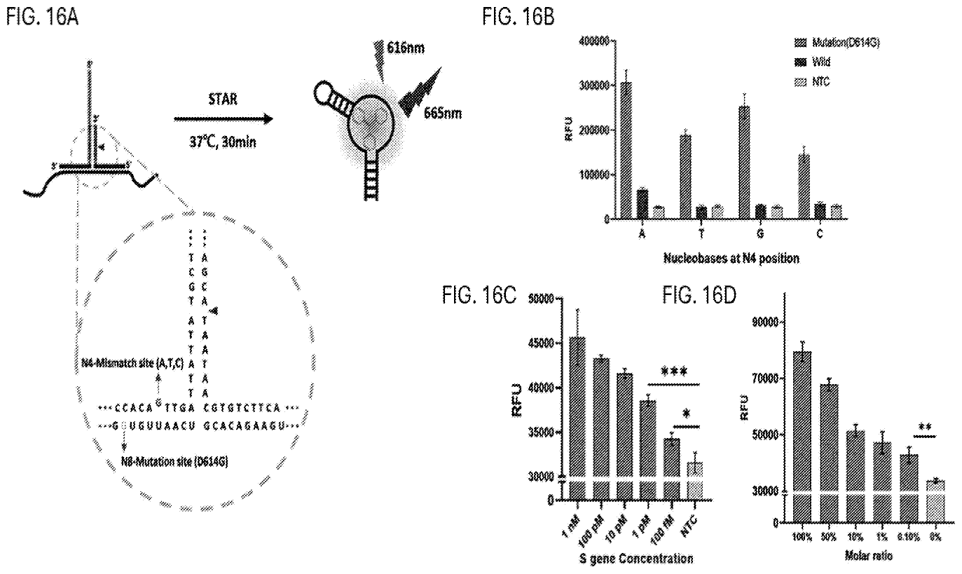

For mutation detection, different nucleobases (A, T, G, and C) at position 4 (N4) were evaluated to find the best one that imparted effective distinction of the mutant (D614G) from the wild target. As a result, the results in FIG. 16B show that G at position N4 produced a high fluorescence signal in the presence of the mutant target (D614G) while minimizing background noise in the presence of the wild target.

In the probe set of the present invention, a third probe of the isothermal one-pot reaction probe set for detecting a mutation may be any one of SEQ ID NOs: 45 to 48, and a fourth probe may be a probe consisting of SEQ ID NO: 49. Preferably, the isothermal one-pot reaction probe set for detecting a mutant may specifically bind to an S gene and may include a probe set in which the third probe is SEQ ID NO: 45 and the fourth probe is SEQ ID NO: 49.

Furthermore, simultaneous detection of the D614G mutation and the N gene of SARS-CoV-2 in one tube was tested, in which a mango aptamer and a malachite green aptamer allowed the N gene and D614G of SARS-CoV-2 to emit fluorescence without interference, respectively (FIG. 17C). It was confirmed that targets for the N gene and D614G could be analyzed simultaneously up to 100 fM, so that highly sensitive detection is possible (FIG. 17D). Therefore, when the probe set of the present invention is used, sensitive detection of SARS-CoV-2 and/or a mutant thereof is possible.

The present invention also provides a composition for detecting SARS-CoV-2 and/or detecting a mutant thereof, including the probe set.

The composition for detecting SARS-CoV-2 and/or detecting a mutant thereof may further include tools and/or reagents commonly used for the detection and/or analysis of a target molecule, in addition to nucleic acid sequences and peptides that specifically bind to SARS-CoV-2 and/or a mutant thereof. These tools or reagents may include suitable carriers, labels capable of generating a detectable signal, solubilizers, detergents, buffers, stabilizers, and the like. When a labeling substance is a fluorescent substance, a substrate needed to measure fluorescence intensity may further be included. Suitable carriers may include soluble carriers, for example, physiologically acceptable buffers known in the art (e.g., PBS, etc.); insoluble carriers, for example, polystyrene, polyethylene, polypropylene, polyester, polyacrylonitrile, fluororesins, cross-linked dextran, polysaccharides, and the like; polymers such as magnetic particles plated with a metal on latex, and paper, glass, metal, agarose, and/or combinations thereof.

The present invention also provides a kit for detecting SARS-CoV-2 and/or detecting a mutant thereof, including the composition for detecting SARS-CoV-2 and/or detecting a mutant thereof, a polymerase, and an isothermal one-pot reaction solution.

The kit of the present invention described above in the present specification may further include various polynucleotide molecules and enzymes, and various buffers and reagents. In addition, the kit of the present invention may include reagents necessary for carrying out positive control and negative control reactions. An optimal amount of reagent to be used in any one particular reaction may be easily determined by one skilled in the art upon learning the disclosure in the present specification.

Since the kit of the present invention is manufactured to perform detection of SARS-CoV-2 and/or detection of a mutant thereof using the probe set of the present invention described above, description of duplicate content is omitted to avoid complexity of the present specification.

In addition, the present invention provides a method of detecting SARS-CoV-2 and/or detecting a mutant thereof, using the isothermal one-pot reaction probe set for detecting SARS-CoV-2 and/or detecting a mutant thereof, and specifically, the method may include the following steps:
(a) treating a sample with an isothermal one-pot reaction probe set for detecting SARS-CoV-2 and/or detecting a mutant thereof consisting of a third probe and a fourth probe of the present invention to bind to or hybridize with SARS-CoV-2 and/or a mutant thereof;
(b) treating a hybridization product of Step (a) with a polymerase to initiate transcription; and
(c) treating a transcription product of Step (b) with an aptamer-reactive substance to detect signal generation of an aptamer in the transcription product.

At this time, the signal generation in Step (c) is characterized in that it indicates the presence of SARS-CoV-2 and/or detecting a mutant thereof in the sample.

Therefore, the method of detecting SARS-CoV-2 and/or detecting a mutant thereof using the probe set of the present invention is designed so that a hybridization step, a transcription reaction, and an aptamer signal reaction occur simultaneously, and Steps (a) to (c) of the present invention may be simultaneously performed in one container.

In addition, an aptamer used to confirm a final signal is not limited to a malachite green aptamer, and any type of RNA-based fluorescent aptamer may be used.

In one embodiment of the present invention, an isothermal nucleic acid amplification reaction may be performed prior to the method of detecting SARS-CoV-2 and/or detecting a mutant thereof.

The type of isothermal nucleic acid amplification reaction is not particularly limited, but in one embodiment of the present invention, the isothermal nucleic acid amplification reaction may be RPA.

It was confirmed that when RPA was performed before Step (a), the signal was amplified compared to when the STAR reaction of the present invention was performed (see FIG. 13). Therefore, in connection with the present invention, an isothermal nucleic acid amplification reaction may be performed as a method of detecting SARS-CoV-2 and/or a mutant thereof more sensitively.

The present invention also provides an on-site molecular diagnostic method using the probe set. The on-site molecular diagnostic method may include the following steps:
(a) treating a sample with an isothermal one-pot reaction probe set for detecting SARS-CoV-2 and/or detecting a mutant thereof consisting of a third probe and a fourth probe to bind to or hybridize with SARS-CoV-2 and/or a mutant thereof;
(b) treating a hybridization product of Step (a) with a polymerase to initiate transcription; and
(c) treating a transcription product of Step (b) with an aptamer-reactive substance to detect signal generation of an aptamer in the transcription product.

At this time, the signal generation in Step (c) is characterized in that it indicates the presence of SARS-CoV-2 and/or a mutant thereof in the sample.

Therefore, a method for detecting a target molecule using a probe set of the present invention may act as a powerful diagnostic method for detecting a target molecule of interest, and it may provide short diagnosis time, high sensitivity and specificity, and simple analysis procedures, and since it does not require expensive equipment or diagnostic experts, it may also be a diagnostic method suitable for new infectious diseases that require rapid response.

Hereinafter, the present invention will be described in more detail through examples. These examples are only for illustrating the present invention, and it will be obvious to those skilled in the art that the scope of the present invention should not be construed as limited by these examples.

### Preparation Example

### Preparation of Reagents and Materials

All DNA sequences were synthesized by Bionics Co. Ltd. (Seoul, Korea). All plasmids were purchased from Integrated DNA Technology Inc. (Skokie, IL, USA). The T7 RNA polymerase, RNase inhibitor, T4 gene 32 protein (single-strand binding protein), and ribonucleotide (rNTP) solution were purchased from Enzynomics Co. Ltd. (Daejeon, Korea). HiScribe T7 High Yield RNA Synthesis Kit, Monarch RNA Cleanup Kit, and DNase I were purchased from New England Biolabs Inc. (pswich, GA, USA). The Bacterial RNA kit was purchased from OMNI International Inc. (Kennesaw, NY, USA). Tris-HCl and MgCl₂ were purchased from Biosesang Co. Ltd. (Seongnam, Korea). Dithiothreitol (DTT) was purchased from ThermoFisher Scientific Inc. (Seoul, Korea). The TO1-3PEG-biotin fluorophore (TOl-biotin) was purchased from Applied Biological Materials Inc. (Richmond, Canada). Malachite green chloride and spermidine were purchased from Sigma-Aldrich Co. Ltd. (St. Louis, MO, USA). All experiments were performed with diethyl pyrocarbonate (DEPC)-treated water to prevent RNA degradation.

### Preparation of DNA Oligonucleotides

The sequences of the oligonucleotides used in the present invention are as shown below.

TABLE 1

| | | SEQ ID |
|---|---|---|
| DNA Probe | Sequence (5′→3′) | NO. |
| Split promoter template | GTACGACAACTACCCCATACCAAACCTTCCTTC | 1 |
| | GTACTTA AAA ATC CCG GCC AGA TTT TGC CAA | |
| | TCA CCC TAT AGT GAG TCG TAT TA GTTAGA | |
| | TTT TGC CAA TCA | |
| Split promoter 1-0 | TTGGCAAAATCTAAC | 2 |
| Split promoter 2-20 | TAATACGACTCACTATAGGG | 3 |
| Split promoter 1-1 | TTGGCAAAATCTAACT | 4 |
| Split promoter 2-19 | AATACGACTCACTATAGGG | 5 |
| Split promoter 1-2 | TTGGCAAAATCTAACTA | 6 |
| Split promoter 2-18 | ATACGACTCACTATAGGG | 7 |
| Split promoter 1-3 | TTGGCAAAATCTAACTAA | 8 |
| Split promoter 2-17 | TACGACTCACTATAGGG | 9 |
| Split promoter 1-4 | TTGGCAAAATCTAACTAAT | 10 |
| Split promoter 2-16 | ACGACTCACTATAGGG | 11 |
| Split promoter 1-5 | TTGGCAAAATCTAACTAATA | 12 |
| Split promoter 2-15 | CGACTCACTATAGGG | 13 |

N gene of SARS-CoV-2

| | | |
|---|---|---|
| Split T7-16 (ST-16)- | ACGACTCACTATAGGG | 14 |
| Universal | | |
| Overlap 0-ST | GTACGACAACTACCCCATACCAAACCTTCCTTC | 15 |
| | GTACCCCTATAGTGAGTCGTATTATGCCAGCC | |
| | ATTCTAG | |
| Overlap 0-ST-4 | CAGCATCACCGCCATTAAT | 16 |
| Overlap 1-ST | GTACGACAACTACCCCATACCAAACCTTCCTTC | 17 |
| | GTACCCCTATAGTGAGTCGTATTATTGCCAGC | |
| | CATTCTAG | |
| Overlap 1-ST-4 | CAGCATCACCGCCATATAAT | 18 |
| Overlap 2-ST | GTACGACAACTACCCCATACCAAACCTTCCTTC | 19 |
| | GTACCCCTATAGTGAGTCGTATTATTTGCCAG | |
| | CCATTCTAG | |
| Overlap 2-ST-4 | CAGCATCACCGCCATAATAAT | 20 |
| Overlap 3-ST | GTACGACAACTACCCCATACCAAACCTTCCTTC | 21 |
| | GTACCCCTATAGTGAGTCGTATTATTTTGCCAG | |
| | CCATTCTAG | |
| Overlap 3-ST-4 | CAGCATCACCGCCATAAATAAT | 22 |
| Overlap 4-ST | GTACGACAACTACCCCATACCAAACCTTCCTTC | 23 |
| | GTACCCCTATAGTGAGTCGTATTATTTTTGCCA | |
| | GCCATTCTAG | |
| Overlap 4-ST-4 | CAGCATCACCGCCATAAAATAAT | 24 |
| Bulge 1-ST | GTACGACAACTACCCCATACCAAACCTTCCTTC | 25 |
| | GTACCCCTATAGTGAGTCGTATTATTTTGCCAG | |
| | CCATTCTAG | |
| Bulge 1-ST-4 | CAGCATCACCGCCATTAATAAT | 26 |
| Bulge 2-ST | GTACGACAACTACCCCATACCAAACCTTCCTTC | 27 |
| | GTACCCCTATAGTGAGTCGTATTATTTTTGCCA | |
| | GCCATTCTAG | |
| Bulge 2-ST-4 | CAGCATCACCGCCATTTAATAAT | 28 |
| Bulge 3-ST | GTACGACAACTACCCCATACCAAACCTTCCTTC | 29 |
| | GTACCCCTATAGTGAGTCGTATTATTTTTTGCC | |
| | AGCCATTCTAG | |
| Bulge 3-ST-4 | CAGCATCACCGCCATTTTAATAAT | 30 |
| Bulge 4-ST | GTACGACAACTACCCCATACCAAACCTTCCTTC | 31 |
| | GTACCCCTATAGTGAGTCGTATTATTTTTTTGC | |
| | CAGCCATTCTAG | |
| Bulge 4-ST-4 | CAGCATCACCGCCATTTTTAATAAT | 32 |
| Bulge 5-ST | GTACGACAACTACCCCATACCAAACCTTCCTTC | 33 |
| | GTACCCCTATAGTGAGTCGTATTATTTTTTTTG | |
| | CCAGCCATTCTAG | |
| Bulge 5-ST-4 | CAGCATCACCGCCATTTTTTAATAAT | 34 |
| Locus 1-ST | GTACGACAACTACCCCATACCAAACCTTCCTTC | 35 |
| | GTACCCCTATAGTGAGTCGTATTATTATGAGG | |
| | AACGAGAAG | |
| Locus 1-ST-4 | TGTTGCGACTACGTGAATAAT | 36 |
| Locus 2-ST | GTACGACAACTACCCCATACCAAACCTTCCTTC | 37 |
| | GTACCCCTATAGTGAGTCGTATTATTTGCCAG | |
| | CCATTCTAG | |
| Locus 2-ST-4 | CAGCATCACCGCCATAATAAT | 38 |
| Locus 3-ST | GTACGACAACTACCCCATACCAAACCTTCCTTC | 39 |
| | GTACCCCTATAGTGAGTCGTATTATTGCT TTA | |
| | GTG GCA GTA | |
| Locus 3-ST-4 | TGT GTT ACA TTG TATTAAT | 40 |
| Locus 4-ST | GTACGACAACTACCCCATACCAAACCTTCCTTC | 41 |
| | GTACCCCTATAGTGAGTCGTATTATTTGTTACA | |
| | TTGTATGC | |
| Locus 4-ST-4 | TCTGCCGAAAGCTTGAATAAT | 42 |
| Locus 5-ST | GTACGACAACTACCCCATACCAAACCTTCCTTC | 43 |
| | GTACCCCTATAGTGAGTCGTATTATTAGTTCCT | |
| | GGTCCCCA | |
| Locus 5-ST-4 | GTTCCTTGTCTGATTAATAAT | 44 |

S gene (D614G Mutation) of SARS-CoV-2

| | | |
|---|---|---|
| Signal template-G | GGATCCATTCGTTACCTGGCTCTCGCCAGTCGGG | 45 |
| | ATCCCCCTATAGTGAGTCGTATTATTAGTTGAC | |
| | ACCCTGAT | |
| Signal template-A | GGATCCATTCGTTACCTGGCTCTCGCCAGTCGGG | 46 |
| | ATCCCCCTATAGTGAGTCGTATTATTAGTTAAC | |
| | ACCCTGAT | |
| Signal template-T | GGATCCATTCGTTACCTGGCTCTCGCCAGTCGGG | 47 |
| | ATCCCCCTATAGTGAGTCGTATTATTAGTTTAC | |
| | ACCCTGAT | |
| Signal template-C | GGATCCATTCGTTACCTGGCTCTCGCCAGTCGGG | 48 |
| | ATCCCCCTATAGTGAGTCGTATTATTAGTTCAC | |
| | ACCCTGAT | |
| D614G Mutation ST-4 | CAGGGACTTCTGTGCAATAAT | 49 |

16S rRNA of Escherichia coli and Staphylococcus aureus

| | | |
|---|---|---|
| E. coli L1-ST | GTACGACAACTACCCCATACCAAACCTTCCTTC | 50 |
| | GTACCCCTATAGTGAGTCGTATTATTCGGGTA | |
| | ACGTCAATG | |
| E. coli L1-ST-4 | CCGGTGCTTCTTCTGAATAAT | 51 |
| E. coli L2-ST | GTACGACAACTACCCCATACCAAACCTTCCTTC | 52 |
| | GTACCCCTATAGTGAGTCGTATTATTCCACGCT | |
| | TTCGCACC | |
| E. coli L2-ST-4 | AATCCTGTTTGCTCCAATAAT | 53 |
| S. aureus L1-ST | GTACGACAACTACCCCATACCAAACCTTCCTTC | 54 |
| | GTACCCCTATAGTGAGTCGTATTATTCGCTTTC | |
| | GCACATCA | |
| S. aureus L1-ST-4 | CCTGTTTGATCCCCAAATAAT | 55 |
| S. aureus L2-ST | GTACGACAACTACCCCATACCAAACCTTCCTTC | 56 |
| | GTACCCCTATAGTGAGTCGTATTATTGGACTT | |
| | AACCCAACA | |
| S. aureus L2-ST-4 | GTTGCGCTCGTTGCGAATAAT | 57 |

The underlined part indicates a mango aptamer sequence and an MG aptamer sequence, and the part in bold indicates a T7 promoter region. In the S gene (D614G Mutation) of SARS-CoV-2, the underlined, bold, and italicized part indicates a mutation site.

TABLE 2

| | | SEQ |
|---|---|---|
| | | ID |
| DNA Probe | Sequence (5′→3′) | NO. |
| SARS-CoV-2 N | TAATACGACTCACTATAGGG | 58 |
| gene_IVT-FP | GCAGTCAAGCCTCTTCTCGT | |
| SARS-CoV-2 N | AACATTGGCCGCAAATTGCA | 59 |
| gene_IVT-RP | | |
| SARS-CoV-1_IVT-FP | TAATACGACTCACTATAGGG | 60 |
| | GCAGTCAAGCCTCTTCTCGC | |
| SARS-CoV-1_IVT-RP | GGTGTGACTTCCATGCCAAT | 61 |
| MERS-CoV_IVT-FP | TAATACGACTCACTATAGGG | 62 |
| | ATAGTCAATCATCTTCAAGA | |
| MERS-CoV_IVT-RP | TTCTGATGGGTAAGTTTAAA | 63 |
| HCoV-229E-FP | TAATACGACTCACTATAGGG | 64 |
| | GTGCTCCTTCCCGGTCTCAG | |
| HCoV-229E-RP | CTTCCAAAGTTGTGGTCAAG | 65 |
| HCoV-NL63-FP | TAATACGACTCACTATAGGG | 66 |
| | GCTCTAATAACTCATCTCGT | |
| HCoV-NL63-RP | TCCCCCATATTGTGATTAAA | 67 |
| HCoV-OC43-FP | TAATACGACTCACTATAGGG | 68 |
| | CTGCTCCTAATTCCAGATCT | |
| HCoV-OC43-RP | AACTCTAATCTTGATCCAAA | 69 |
| HCoV-HKU1-FP | TAATACGACTCACTATAGGG | 70 |
| | CTGCTTCTAATAGTCGACCA | |
| HCoV-HKU1-RP | AAGTCTAATTTAGAACCAAA | 71 |
| SARS-CoV-2 S | TAATACGACTCACTATAGGG | 72 |
| gene | TCTAACCAGGTTGCTGTTCT | |
| (G614)_IVT-FP | TTATCAGGGTGTTAACTGCA | |
| | CAGAAGTCCC | |
| SARS-CoV-2 S gene | GGAGTAAGTTGATCTGCATG | 73 |
| (G614)_IVT-RP | AATAGCAACAGGGACTTCTG | |
| | TGCAGTTAAC | |
| SARS-CoV-2 S gene | TAATACGACTCACTATAGGG | 74 |
| (D614)_IVT-FP | TCTAACCAGGTTGCTGTTCT | |
| | TTATCAGGATGTTAACTGCA | |
| | CAGAAGTCCC | |
| SARS-CoV-2 S gene | GGAGTAAGTTGATCTGCATG | 75 |
| (D614)_IVT-RP | AATAGCAACAGGGACTTCTG | |
| | TGCAGTTAAC | |

The part in bold indicates a T7 promoter region. In the S gene (G614, D614G Mutation) of SARS-CoV-2, the underlined, bold, and italicized part indicates a mutation site.

### Preparation of Target RNA Sequence

Target RNAs were generated through in vitro transcription. The sequence information of the target RNAs used in the present invention is shown in [Table 3].

TABLE 3

| | | | SEQ |
|---|---|---|---|
| | | Size | ID |
| Name | Sequence (5′→3′) | (bp) | NO. |
| SARS-CoV- | GCAGUCAAGCCUCUUCUCGUUCCUCAUCACGUAGU | 380 | 76 |
| 2 N gene | CGCAACAGUUCAAGAAAUUCAACUCCAGGCAGCAG | | |
| | UAGGGGAACUUCUCCUGCUAGAAUGGCUGGCAAUG | | |
| | GCGGUGAUGCUGCUCUUGCUUUGCUGCUGCUUGAC | | |
| | AGAUUGAACCAGCUUGAGAGCAAAAUGUCUGGUAA | | |
| | AGGCCAACAACAACAAGGCCAAACUGUCACUAAGA | | |
| | AAUCUGCUGCUGAGGCUUCUAAGAAGCCUCGGCAA | | |
| | AAACGUACUGCCACUAAAGCAUACAAUGUAACACA | | |
| | AGCUUUCGGCAGACGUGGUCCAGAACAAACCCAAG | | |
| | GAAAUUUUGGGGACCAGGAACUAAUCAGACAAGGA | | |
| | ACUGAUUACAAACAUUGGCCGCAAAUUGCA | | |
| SARS-CoV- | GCAGUCAAGCCUCUUCUCGCUCCUCAUCACGUAGU | 442 | 77 |
| 1 | CGCGGUAAUUCAAGAAAUUCAACUCCUGGCAGCAG | | |
| | UAGGGGAAAUUCUCCUGCUCGAAUGGCUAGCGGAG | | |
| | GUGGUGAAACUGCCCUCGCGCUAUUGCUGCUAGAC | | |
| | AGAUUGAACCAGCUUGAGAGCAAAGUUUCUGGUAA | | |
| | AGGCCAACAACAACAAGGCCAAACUGUCACUAAGA | | |
| | AAUCUGCUGCUGAGGCAUCUAAAAAGCCUCGCCAA | | |
| | AAACGUACUGCCACAAAACAGUACAACGUCACUCA | | |
| | AGCAUUUGGGAGACGUGGUCCAGAACAAACCCAAG | | |
| | GAAAUUUCGGGGACCAAGACCUAAUCAGACAAGGA | | |
| | ACUGAUUACAAACAUUGGCCGCAAAUUGCACAAUU | | |
| | UGCUCCAAGUGCCUCUGCAUUCUUUGGAAUGUCAC | | |
| | GCAUUGGCAUGGAAGUCACACC | | |
| MERS-CoV | AUAGUCAAUCAUCUUCAAGAGCCUCUAGCUUAAGC | 451 | 78 |
| | AGAAACUCUUCCAGAUCUAGUUCACAAGGUUCAAG | | |
| | AUCAGGAAACUCUACCCGCGGCACUUCUCCAGGUC | | |
| | CAUCUGGAAUCGGAGCAGUAGGAGGUGAUCUACUU | | |
| | UACCUUGAUCUUCUGAACAGACUACAAGCCCUUGA | | |
| | GUCUGGCAAAGUAAAGCAAUCGCAGCCAAAAGUAA | | |
| | UCACUAAGAAAGAUGCUGCUGCUGCUAAAAAUAAG | | |
| | AUGCGCCACAAGCGCACUUCCACCAAAAGUUUCAA | | |
| | CAUGGUGCAAGCUUUUGGUCUUCGCGGACCAGGAG | | |
| | ACCUCCAGGGAAACUUUGGUGAUCUUCAAUUGAAU | | |
| | AAACUCGGCACUGAGGACCCACGUUGGCCCCAAAU | | |
| | UGCUGAGCUUGCUCCUACAGCCAGUGCUUUUAUGG | | |
| | GUAUGUCGCAAUUUAAACUUACCCAUCAGAA | | |
| HCoV-229E | GUGCUCCUUCCCGGUCUCAGUCGAGGUCGCAGAGU | 391 | 79 |
| | CGCGGUCGUGGUGAAUCCAAACCUCAAUCUCGGAA | | |
| | UCCUUCAAGUGACAGAAACCAUAACAGUCAGGAUG | | |
| | ACAUCAUGAAGGCAGUUGCUGCGGCUCUUAAAUCU | | |
| | UUAGGUUUUGACAAGCCUCAGGAAAAAGAUAAAAA | | |
| | GUCAGCGAAAACGGGUACUCCUAAGCCUUCUCGUA | | |
| | AUCAGAGUCCUGCUUCUUCUCAAACUUCUGCCAAG | | |
| | AGUCUUGCUCGUUCUCAGAGUUCUGAAACAAAAGA | | |
| | ACAAAAGCAUGAAAUGCAAAAGCCACGGUGGAAAA | | |
| | GACAGCCUAAUGAUGAUGUGACAUCUAAUGUCACA | | |
| | CAAUGUUUUGGCCCCAGAGACCUUGACCACAACUU | | |
| | UGGAAG | | |
| HCoV-NL63 | GCUCUAAUAACUCAUCUCGUGCUAGCAGUCGUUCU | 355 | 80 |
| | UCAACUCGUAACAACUCACGAGACUCUUCUCGUAG | | |
| | CACUUCAAGACAACAGUCUCGCACUCGUUCUGAUU | | |
| | CUAACCAGUCUUCUUCAGAUCUUGUUGCUGCUGUU | | |
| | ACUUUGGCUUUAAAGAACUUAGGUUUUGAUAACCA | | |
| | GUCGAAGUCACCUAGUUCUUCUGGUACUUCCACUC | | |
| | CUAAGAAACCUAAUAAGCCUCUUUCUCAACCCAGG | | |
| | GCUGAUAAGCCUUCUCAGUUGAAGAAACCUCGUUG | | |
| | GAAGCGUGUUCCUACCAGAGAGGAAAAUGUUAUUC | | |
| | AGUGCUUUGGUCCUCGUGAUUUUAAUCACAAUAUG | | |
| | GGGGA | | |
| HCoV-OC43 | CUGCUCCUAAUUCCAGAUCUACUUCGCGCACAUCC | 412 | 81 |
| | AGCAGAGCCUCUAGUGCAGGAUCGCGUAGUAGAGC | | |
| | CAAUUCUGGCAAUAGAACCCCUACCUCUGGUGUAA | | |
| | CACCUGACAUGGCUGAUCAAAUUGCUAGUCUUGUU | | |
| | CUGGCAAAACUUGGCAAGGAUGCCACUAAACCUCA | | |
| | GCAAGUAACUAAGCAUACUGCCAAAGAAGUCAGAC | | |
| | AGAAAAUUUUGAAUAAGCCCCGCCAGAAGAGGAGC | | |
| | CCCAAUAAACAAUGCACUGUUCAGCAGUGUUUUGG | | |
| | UAAGAGAGGCCCUAAUCAGAAUUUUGGUGGUGGAG | | |
| | AAAUGUUAAAACUUGGAACUAGUGACCCACAGUUC | | |
| | CCCAUUCUUGCAGAACUCGCACCCACAGCUGGUGC | | |
| | GUUUUUCUUUGGAUCAAGAUUAGAGUU | | |
| HCoV- | CUGCUUCUAAUAGUCGACCAGGUUCACGUUCUCAA | 409 | 82 |
| HKU1 | UCACGUGGACCCAAUAAUCGUUCAUUAAGUAGAAG | | |
| | UAAUUCUAAUUUUAGACAUUCAGAUUCUAUAGUAA | | |
| | AACCUGAUAUGGCUGAUGAGAUCGCUAAUCUUGUU | | |
| | UUAGCCAAGCUUGGUAAAGAUUCUAAACCUCAGCA | | |
| | AGUCACUAAGCAAAAUGCCAAGGAAAUCAGGCAUA | | |
| | AAAUUUUAACAAAACCUCGCCAAAAGCGAACUCCU | | |
| | AAUAAACAUUGUAAUGUUCAACAGUGUUUUGGUAA | | |
| | AAGAGGACCUUCUCAAAAUUUUGGUAAUGCUGAAA | | |
| | UGUUAAAGCUUGGUACUAAUGAUCCUCAGUUUCCU | | |
| | AUUCUUGCAGAAUUAGCUCCUACACCAGGUGCUUU | | |
| | UUUCUUUGGUUCUAAAUUAGACUU | | |
| SARS-CoV- | UCUAACCAGGUUGCUGUUCUUUAUCAGGGUGUUAA | 80 | 83 |
| 2 S gene | CUGCACAGAAGUCCCUGUUGCUAUUCAUGCAGAUC | | |
| (G614) | AACUUACUCC | | |
| SARS-CoV- | UCUAACCAGGUUGCUGUUCUUUAUCAGGAUGUUAA | 80 | 84 |
| 2 S gene | CUGCACAGAAGUCCCUGUUGCUAUUCAUGCAGAUC | | |
| (D614) | AACUUACUCC | | |
| E. coli | AGACUCCUACGGGAGGCAGCAGUGGGGAAUAUUGC | 610 | 85 |
| 16S rRNA | ACAAUGGGCGCAAGCCUGAUGCAGCCAUGCNGCGU | (327- | |
| | GUAUGAAGAAGGCCUUCGGGUUGUAAAGUACUUUC | 936) | |
| | AGCGGGGAGGAAGGGAGUAAAGUUAAUACCUUUGC | | |
| | UCAUUGACGUUACCCGCAGAAGAAGCACCGGCUAA | | |
| | CUCCGUGCCAGCAGCCGCGGUAAUACGGAGGGUGC | | |
| | AAGCGUUAAUCGGAAUUACUGGGCGUAAAGCGCAC | | |
| | GCAGGCGGUUUGUUAAGUCAGAUGUGAAAUCCCCG | | |
| | GGCUCAACCUGGGAACUGCAUCUGAUACUGGCAAG | | |
| | CUUGAGUCUCGUAGAGGGGGGUAGAAUUCCAGGUG | | |
| | UAGCGGUGAAAUGCGUAGAGAUCUGGAGGAAUACC | | |
| | GGUGGCGAAGGCGGCCCCCUGGACGAAGACUGACG | | |
| | CUCAGGUGCGAAAGCGUGGGGAGCAAACAGGAUUA | | |
| | GAUACCCUGGUAGUCCACGCCGUAAACGAUGUCGA | | |
| | CUUGGAGGUUGUGCCCUUGAGGCGUGGCUUCCGGA | | |
| | NNUAACGCGUUAAGUCGACCGCCUGGGGAGUACGG | | |
| | CCGCAAGGUUAAAACUCAAAUGAAUUGACGGGGGC | | |
| | CGCACAAGCGGUGGA | | |
| S. aureus | CAGAAGAGGAAAGUGGAAUUCCAUGUGUAGCGGUG | 524 | 86 |
| 16S rRNA | AAAUGCGCAGAGAUAUGGAGGAACACCAGUGGCGA | (666- | |
| | AGGCGACUUUCUGGUCUGUAACUGACGCUGAUGUG | 1188) | |
| | CGAAAGCGUGGGGAUCAAACAGGAUUAGAUACCCU | | |
| | GGUAGUCCACGCCGUAAACGAUGAGUGCUAAGUGU | | |
| | UAGGGGGUUUCCCGCCCCUUAGUGCUGCAGCUAAC | | |
| | GCAUUAAGCACUCCGCCUGGGGAGUACGACCGCAA | | |
| | GGUUGAAACUCAAAGGAAUUGACGGGGACCCGCAC | | |
| | AAGCGGUGGAGCAUGUGGUUUAAUUCGAAGCAACG | | |
| | CGAAGAACCUUACCAAAUCUUGACAUCCUUUGACA | | |
| | ACUCUAGAGAUAGAGCCUUCCCCUUCGGGGGACAA | | |
| | AGUGACAGGUGGUGCAUGGUUGUCGUCAGCUCGUG | | |
| | UCGUGAGAUGUUGGGUUAAGUCCCGCAACGAGCGC | | |
| | AACCCUUAAGCUUAGUUGCCAUCAUUAAGUUGGGC | | |
| | ACUCUAAGUUGACUGCCGGUGACAAACCGGAGGA | | |

### Example 1

### Optimization of Split T7 Promoter-Based DNA Probes and Reaction Conditions

### 1-1. T7 Promoter Split Ratio

Since the core of the present invention is the effective formation of a split T7 promoter in the presence of a target molecule by inducing a three-way junction structure, an existing T7 promoter was split, and the transcription efficiency according to each ratio was analyzed before the present invention (FIG. 3).

To confirm transcription with the split T7 promoters, a transcription reaction was performed using three types of DNA probes, which were Split Promoter 1 (SP1), Split Promoter 2 (SP2), and Template (T). The reaction mixture consisted of 2 μL of SP1, SP2, and T (1 μM each), 2 μL of 10×T7 buffer, 0.8 μL of T7 RNA polymerase (2 U/μL), 0.4 μL of RNase inhibitor (0.08 U/μL), and 2 μL of TO1-biotin (1 μM), 1 μL of rNTPs (2.5 mM each), and 11.8 μL of DEPC-treated water. The reaction mixture was incubated at 37° C. for one hour and then transferred to a 384-well plate. Fluorescence signals were measured at excitation and emission wavelengths of 507 nm and 547 nm, respectively, using a microplate reader (SpectraMax iD5 Multi-Mode Microplate Reader, Molecular Devices, USA).

Target RNAs were produced through in vitro transcription. The sequence information of the target RNAs used in the present invention is shown in Table 3 above.

Specifically, using each plasmid as a PCR template and a primer including a T7 promoter sequence, a DNA sequence corresponding to a target RNA region was first produced. Next, a transcription reaction solution consisting of 1 μg of template DNA, 1.5 μL of 10× reaction buffer (final 0.75×), 1.5 μL of rNTP (7.5 mM each), 1.5 μL of T7 RNA polymerase Mix, and 14.5 μL of DEPC-treated water was prepared. Then, the transcription reaction was performed by incubation at 37° C. for 16 hours, after which a PCR product was digested with DNase I. The RNA transcription product was purified using the Monarch RNA Cleanup Kit, quantified by NanoDrop at a wavelength of 260 nm, and stored at −80° C. until use.

To control the formation of a T7 promoter, nick sites were sequentially inserted starting from the 5′ end of the T7 promoter to prepare T7 promoters split at various ratios. After transcription by placing a complementary sequence of the mango aptamer on the template, the transcription reaction was monitored by measuring the fluorescence signal of TO-1 biotin. As the sequence of the split T7 promoter DNA probe, the above-described sequences of SEQ ID NOs: 1 to 13 in [Table 1] were used.

As a result, as shown in FIG. 3, addition of T+SP2 or T+SP1+SP2 in the complete T7 promoter (0:20 split ratio) resulted in efficient transcription as indicated by a high fluorescence signal. When a split T7 promoter at a 1:19 ratio was used, the signal was increased compared to the complete T7 promoter even in the presence of a nick site (T+SP1+SP2). In addition, transcription occurred effectively in the presence of T+SP2. When the split ratio was changed from 2:18 to 4:16, transcription occurred effectively in the presence of T+SP1+SP2. After the ratio of 3:17, the fluorescence value drastically decreased due to the absence of SP1, indicating that the 3-nucleotide region on the 5′ side of the T7 promoter is important for the T7 RNA polymerase to specifically recognize the T7 promoter. In addition, in the case of 5:15, it was confirmed that almost no transcription occurred even when both SP1 and SP2 were present, indicating that when a nick site is present after the 5-mer on the 5′ side of the T7 promoter, the T7 RNA polymerase is unable to normally recognize the T7 promoter.

It was confirmed that a split ratio of 4:16 is most suitable to form a complete double-stranded T7 promoter through the formation of a three-way junction structure. 1-2. Optimization of overlapping sequence Afterward, structural optimization was performed to apply the T7 promoter at a 4:16 ratio to the 3-way junction structure (FIG. 4).

The 16-nucleotides on the 3′ side of the T7 promoter bind complementarily to the signal template and are universal. Only when a target RNA is present, since a first probe consisting of ST and split T7-16 (Split T7-16, ST-16) and a second probe (Split T7-4, ST-4) need to be combined to form a complete T7 promoter with a nick site, a Tm value of an overlapping sequence portion is the most important factor, and the Tm value was adjusted by arranging an additional overlapping sequence (0 to 4 bp). The additional overlapping sequence portion basically includes 4 bp of the 5′ portion of the T7 promoter.

The above-described sequences of SEQ ID NOs: 13 to 24 in [Table 1] were used as the DNA probe sequences for optimizing the overlapping sequence.

First, when an overlapping sequence (N=0) was 4 bp of a T7 promoter, no fluorescence was produced when a target sequence was not present, so it is suitable for the purpose of not forming a structure. However, it was confirmed that when a target was present, a three-way junction structure was not completely formed due to excessively short overlapping, so the T7 promoter was not formed, and transcription was not performed effectively. It was confirmed that when an overlapping sequence of 1 bp or more was added (N=1), a high fluorescence value was observed only when a target sequence was present. Through this, it was confirmed that in order to stably form a three-way junction structure and induce a transcriptional reaction, an overlap of at least 1 bp is required in addition to 4 bp from the T7 promoter. When an added overlapping sequence exceeded 3 bp, non-specific transcription occurred even when no target sequence was present.

Therefore, the appropriate additional overlapping sequence was set to 2 bp, and the total overlapping portion was set to 6 bp (4 bp from the T7 promoter).

Afterward, a bulge structure was added to the 3-way junction structure to test the stability of the T7 promoter including a double-stranded nick site (FIG. 5). In the case where a bulge structure was introduced, as the DNA sequences of ST and ST-4, the above-described sequences of SEQ ID NOs: 25 to 34 in [Table 1] were used.

As a result, as shown in FIG. 5, it was confirmed that when a bulge was added, the stability of the structure rather decreases, so a three-way junction structure was not properly formed even when a target nucleic acid sequence was present, resulting in less transcription.

Therefore, it was confirmed that the optimal structure includes no additional overlapping sequences except for the complementary 2 bp, including no formed bulge structure.

### 1-3. Optimization of Reaction Conditions

To obtain the best performance of the proposed STAR, an optimization experiment was performed on a buffer, enzyme, reaction time, and reaction temperature.

The concentration of DTT was set to 0.375 mM to 1.5 mM, and the concentration of MgCl₂ was set to 0 to 20 mM, and each case (25 cases; 5×5) was tested three times to measure the fluorescence intensity. The fluorescence intensity of spermidine was measured in the absence and presence of a target at a concentration of 0 to 5 mM, and the fluorescence intensity of SSB was also measured at a concentration of 0 to 50 ng/μl. The measurement was performed under the conditions of 0 to 80 U of T7 RNA polymerase, a reaction time of 0 to 120 minutes, and a reaction temperature of 25, 30, 37, and 41° C. In addition, in order to optimize the annealing method, results were compared under the conditions of fast cooling, slow cooling, and without cooling (w/o cooling).

As a result, as shown in FIGS. 6 to 9, the ideal conditions were as follows: 10 mM MgCl₂, 1.5 mM DTT (FIG. 6), 3 mM spermidine, 20 ng/μL SSB (FIG. 7), 20 U of T7 RNA polymerase, a reaction time of 30 minutes, and a reaction temperature of 37° C. (FIGS. 8A-8D) without cooling.

Therefore, due to the optimized amount of spermidine and SSB that can mediate efficient hybridization of the probe and target RNA, conditions under which a three-way junction structure may be effectively formed and a transcription reaction may be induced without initial thermal denaturation and cooling steps were confirmed. Therefore, considering the need to perform the entire analysis work under isothermal conditions (37° C.), the present invention is very advantageous in terms of actual on-site application.

### 1-4. Comparison of Transcription Efficiency Between Three-Way Junction Structure and Linear Structure

Finally, to compare the optimized buffer and the three-way junction structure with the existing T7 promoter, real-time fluorescence monitoring was performed using SYBR Green II, which emits a high fluorescence signal by binding to single-stranded RNA.

As a result, as shown in FIG. 9, there was no significant difference compared to the existing T7 promoter, and rather, it was found that the transcription of the three-way junction structure had transcription efficiency almost similar to the transcription of the linear structure.

### 1-5. Performing Gel Electrophoresis to Confirm Three-Way Junction Structure

The present invention is a technology that combines a three-way junction structure-based split T7 promoter and fluorescent RNA aptamer technology, and a fluorescence signal is amplified when a target nucleic acid biomarker is present.

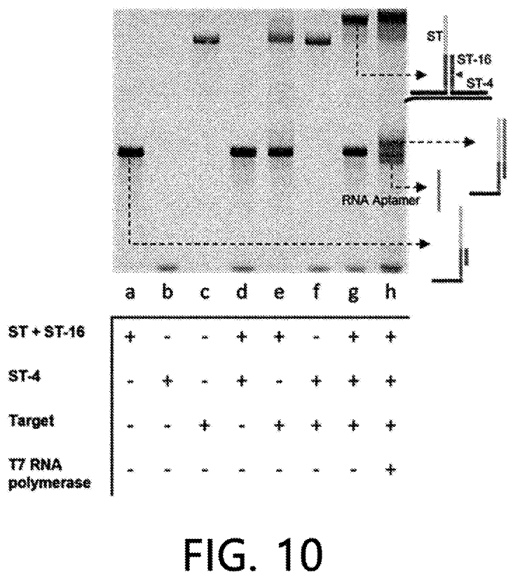

DNA probes are composed of two types of single-stranded DNA. A single template (signal template, ST) consists of a sequence complementary to the mango aptamer, one of the fluorescent RNA aptamers (yellow), a T7 promoter sequence (blue), and a sequence complementary to half of a target sequence (black). Split T7-16 (ST-16) is a partial sequence (16mer; blue) of the T7 promoter and is complementary to the T7 promoter of the ST. It has a Tm value of 56.3° C. and is universal because it binds complementarily to the ST regardless of the target material in a reaction at 37° C. Gel electrophoresis was performed to confirm the three-way junction structure, and FIG. 10 shows the results of the polyacrylamide gel electrophoresis.

The formation of a three-way junction structure was confirmed by polyacrylamide gel electrophoresis (PAGE) using an 8% polyacrylamide gel. Samples were prepared by incubating 1 μM of the signal template (ST), 1 μM of Split T7-16 (ST-16), 1 μM of Split T7-4 (ST-4), 500 nM of N gene RNA, and 0.5 U/μL of T7 RNA polymerase at 37° C. for 30 minutes. The prepared samples were loaded onto the gel, and a 1×TBE buffer was loaded at 200 V for 40 minutes. In addition, agarose gel electrophoresis was performed using a 2% agarose gel in 1×TBE buffer at 135 V for one hour. The results were visualized using a GreenStar nucleic acid staining solution (Bioneer Inc., Daejeon, Korea), and the gels were scanned using ChemiDoc (Bio-Rad Laboratories, Inc., CA, USA).

The split T7-4 (ST-4) consists of a partial sequence of the T7 promoter (4-mer; blue) and a sequence capable of complementarily binding to the other half of the sequence of a target sequence (black). All reactions in the present invention were carried out at 37° C. The 5′-ATTATT-3′ sequence of the ST is complementary to ST-4, but since the Tm value is 12° C., it was designed to prevent binding at 37° C. Therefore, when a target material is not present, the ST and ST-4 probes among the DNA probes may not bind, and thus a three-way junction structure may not be formed, preventing the formation of a complete double-stranded T7 promoter. When a target substance is present, the target RNA preferentially binds to the complementary sequences of the target substance, ST and ST-4, to form a three-way junction structure. Through this, a double-stranded T7 promoter including a nick site is formed.

As a result, as shown in FIG. 9, the formation of a three-way junction structure was confirmed through the PAGE analysis in the presence of a target and a T7 RNA polymerase-catalyzed transcription reaction. As shown in FIG. 9, ST and ST-16 hybridized with each other (a), and ST, ST-16, and ST-4 did not form a three-way junction structure in the absence of the target (d). In addition, when all DNA probes and targets were present (g), the presence of T7 RNA polymerase formed a three-way junction structure that generated a large amount of light-up RNA aptamers through transcription (h).

### 1-6. Evaluation of Fluorescence Emission Spectra Under Various Reaction Conditions

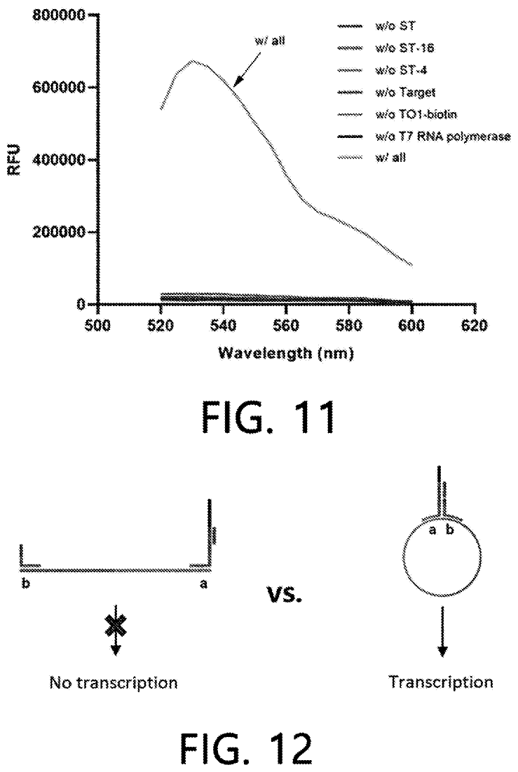

Next, the fluorescence spectrum without each element of STAR was evaluated (FIG. 11). RNA transcription occurred by a T7 RNA polymerase, and a mango aptamer was generated. Finally, To1-biotin, a fluorescent dye that can bind to the mango aptamer, was bound to exhibit a high fluorescence signal. Without one of the DNA probes (ST, ST-16, ST-4) or a target sequence, a double-stranded T7 promoter including a nick site that is necessary for RNA transcription was not formed, and the mango aptamer was not produced. Therefore, binding between the mango aptamer and TO1-biotin did not occur, and a negligible fluorescence signal was exhibited. In addition, no fluorescence signal appears even in the absence of TO1-biotin or T7 RNA polymerase. A high fluorescence signal was generated only when all elements were present (w/all), indicating that all elements of STAR each play an important role in the one-pot detection of a target RNA.

### Example 2

### Measurement of Detection Sensitivity for Each Locus of SARS-CoV-2 N Gene

In the present invention, it was assumed that the efficiency of forming a three-way junction structure, which is the core of STAR, may be different for each target region and lead to different detection sensitivities. SARS-CoV-2 was selected as a target material, and five types of probes were designed for certain regions of the N gene (Nucleotide positions 28809 to 29188) (FIG. 14A). For the sequence of the N gene, the NCBI Accession number NC_045512 was referred to. As the specific sequence information about the five types of probes, the above-described sequences of SEQ ID NOs: 35 to 44 in [Table 1] were used.

After confirming that each region is specific to SARS-CoV-2 through multiple sequence alignment of DNA STAR, a fold change was calculated by dividing the fluorescent signal at 100 μM (6×10⁷ copies/μL) to 100 fM (6×10⁴ copies/μL) in the present of a target (F) by the fluorescent signal in the absence of the target (FNTC: non-target control). As shown in the heatmap in FIG. 14B, transcription efficiency was dependent on the targeted locus. Among the five DNA probe sets (Locus 1 to 5), the DNA probes for L2 and L4 showed higher fold changes than the other probes (L1, L3, and L5) and determined the target N gene up to 100 fM (FIG. 14C). As a result, it was confirmed that the sensitivity was different for each region and that among the five regions, L2 and L4 may be quantitatively detected up to 100 fM.

### Example 3

### Targeting Two Loci to Improve Detection Sensitivity of STAR

In order to improve the sensitivity based on the experimental results for each region (FIGS. 14A-14C), a method of using two probes simultaneously was introduced by targeting various regions of the target gene (N gene) in one reaction. Based on the results of Example 2, STAR probes targeting L2 and L4, which exhibited the best sensitivity, were selected. FIG. 15A shows a schematic diagram of two different loci of the SARS-CoV-2 N gene and of other human coronaviruses, in which the nucleotides of the RNA sequences of other viruses that are different from SARS-CoV-2 are shaded in gray. The sequence information of each DNA probe is as shown in [Table 2] above, and the sequence information of the target RNA is as shown in [Table 3] above.

Each STAR probe generates a signal by binding to each locus in a target RNA region, so the signal is capable of being amplified. To confirm that the STAR probes in both regions (L2 and L4) simultaneously bind to one target, as described above in Example 1-5, agarose gel electrophoresis was first performed. The concentration of the L2 and L4 probe sets was 500 nM each (a and b), and when the L2 and L4 probes were present simultaneously, the concentration of each probe set was 250 nM (c). As a result, as shown in FIG. 15B, it was confirmed that when two probe sets were present simultaneously (c), the electrophoretic mobility was reduced compared to the case where only a single probe set (L2 or L4) was present (a and b), which was confirmed by a higher band position. These results demonstrate that the two STAR probes specifically bind to each region of the target RNA.

For detection of the target RNA, all reagents for STAR were premixed: 4 μl of 1OX STAR buffer solution (400 mM Tris-HCl, 100 mM MgCl₂, 15 mM DTT, 30 mM spermidine), 6 μl of STAR probe set (2 μL of Signal Template (2 μM), 2 μL of Split T7-16 (2 μM), and 2 μL of Split T7-4 (2 μM)), 4 μL of TO1-biotin (1 μM) or 2 μL of malachite green chloride (100 μM), 4 μL of SSB (20 ng/μL), 4 μL of rNTP (2.5 mM each), 0.4 μL of T7 RNA polymerase (0.5 U/μl), 0.8 μL of RNase inhibitor (0.08 U/μL), and DEPC-water (maximum 36 μL). The reagents may be mixed in an arbitrary order until the target RNA is added. Next, 4 μL of the target RNA was added, and the resulting reaction mixture was incubated at 37° C. for 30 minutes. For dual targeting at the N gene (FIG. 15A), 50 nM of each STAR probe set (L2 and L4) was added. Finally, the reaction mixture was transferred to a 384-well plate, where the fluorescence signal corresponding to TO1-biotin binding to the mango aptamer was analyzed using a microplate reader (SpectraMax iD5 Multi-Mode Microplate Reader, Molecular Devices LLC., USA) at the excitation and emission wavelengths of 507 nm and 547 nm, respectively. To measure the fluorescence signal from malachite green binding to the malachite green aptamer, 616 nm and 665 nm were used as excitation and emission wavelengths, respectively.

The fluorescence intensity was compared between the case where two probe sets were used simultaneously and the case where only one probe set was used. The fluorescence signal was measured in the presence of the L2 probe set (100 nM) and the L4 probe set (100 nM), separately, and in the presence of both the L2 and L4 probe sets (50 nM+50 nM), and the concentration of the target N gene RNA was 1 nM. It was confirmed that the fluorescence signal increased when two probe sets were used simultaneously (L2+L4) (FIG. 15C), indicating that the method may be used as a good method for signal improvement, especially in limited situations where the target RNA concentration is low.

The detection limit when using the STAR probe set combination at L2 and L4 was measured. The concentration of the STAR probe set was 50 nM at each different locus. It was found that the target RNA may be determined up to 10² copies/μL by targeting two loci, which is a 600-fold improvement compared to the targeting of one locus, and this is sufficient for screening of SARS-CoV-2 in actual clinical situations (FIG. 15D).

In addition, the specificity of STAR was evaluated with a DNA probe that was designed not to bind to six human coronaviruses, including other alphacoronaviruses and betacoronaviruses. The nucleotides of the six coronaviruses that are different from the SARS-CoV-2 region are shown in gray (FIG. 15A). In the presence of the six coronavirus RNAs at a concentration of 100 nM, the fluorescence signal was similar to that observed for NTC, and a highly enhanced fluorescence signal was generated only for the SARS-CoV-2 N gene at 1 nM (FIG. 15E). This indicates that only the targeted SARS-CoV-2 may be specifically and sensitively detected even in situations where the RNA target concentration is low.

### Example 4

### 4-1. Multiplex STAR for Detecting SARS-CoV-2 D614G Mutation

An experiment was conducted to demonstrate the multi-purpose applicability of STAR by selecting the D614G mutation of SARS-CoV-2 as a target. In addition, multiplex detection for simultaneous detection of the D614G mutation and the N gene of SARS-CoV-2 in a single tube was also confirmed.

The D614G mutation is known to be present in all SARS-CoV-2 variants, including the alpha, beta, and delta variants. In FIG. 16A, the mutation is indicated in yellow, and mismatch sequences indicated in red were added to avoid false positive signals in the wild target. As adenine (A) is replaced by guanine (G) at position 23403 of SARS-CoV-2 genomic RNA, a specific STAR probe was rationally designed to identify this change, and a malachite green aptamer and a fluorogenic dye (malachite green) with a different wavelength were used for a multiplexing experiment (FIG. 16A). As the sequence of the DNA probe, the above-described sequences of SEQ ID NOs: 45 to 49 in [Table 1] were used.

A multiplex analysis was performed using 4 μL of 10×STAR buffer (400 mM Tris-HCl, 100 mM MgCl₂, 15 mM DTT, 30 mM spermidine), 3 μL of an L2 probe set (2 μM each), 3 μL of an L4 probe set (2 μM each), 6 μL of a D614G mutant probe set (2 μM each), 4 μL of TO1-biotin (1 μM), 2 μL of malachite green chloride (100 μM), 4 μL of SSB (20 ng/μL), 4 μL of rNTPs (2.5 mM each), 0.4 μL of T7 RNA polymerase (0.5 U/μL), 0.8 μL of RNase inhibitor (0.08 U/μL), and DEPC-water (maximum 32 μL) and adding 8 μL of target RNA thereto.

Different nucleobases (A, T, G, and C) at position 4 (N4) were evaluated to find the best one that imparted effective distinction of the mutant (D614G) from the wild type, and the results in FIG. 16B showed that G at position N4 generated a high fluorescence signal in the presence of the mutant target (D614G) while minimizing background noise in the presence of the wild target. As the DNA probe sequence, the above-described sequences of SEQ ID NOs: 72 to 75 in [Table 2] were used, and as the target RNA sequence information, the above-described SEQ ID NOs: 83 and 84 in [Table 3] were used.

As a result, a detection limit of 100 fM was obtained for the mutation, and it was confirmed that the wild type did not interfere with mutation detection (FIG. 16C). In addition, mixtures of the mutant/wild target with different molar ratios (0%, 0.1%, 1%, 10%, 50%, and 100%) were analyzed using the proposed STAR.

In addition, FIG. 16D shows that the mutant target at a ratio of 0.1% is distinguished from the wild target, which is similar to or better than other methods, confirming the excellent specificity of the present invention.

### 5-2. System for Multiplex Detection

Multiplex STAR was demonstrated for simultaneous detection of the D614G mutation and N gene of SARS-CoV-2 in one tube, where the mango aptamer and malachite green aptamer correspond to the N gene and D614G of SARS-CoV-2, respectively. FIG. 17A shows a schematic diagram of one-pot multiplex STAR for simultaneous detection of the SARS-CoV-2 N gene and D614G mutation.

Since the mango and malachite green aptamers were used for simultaneous detection, it was confirmed whether the two aptamers emit fluorescence without interference. As a result, FIG. 17B shows the normalized fluorescence emission spectra of TO1-biotin and MG binding to the mango aptamer and MG aptamer, respectively. The fluorescence emission spectra of TO1-biotin were recorded at 520 nm to 570 nm and an excitation wavelength of 480 nm, and the fluorescence emission spectra of malachite green were recorded at 640 nm to 720 nm and an excitation wavelength of 600 nm. It was confirmed that the TO1-biotin and malachite green generated distinct fluorescence emission spectra (green and red) without interference after binding to the mango and malachite green aptamers, respectively (FIG. 17B).

In addition, the STAR probe for the N gene produced a highly enhanced fluorescence signal (green) through the production of the mango aptamer only when the N gene of SARS-CoV-2 was present, and the STAR probe for the D614G mutation generated a highly enhanced fluorescence signal. FIG. 17C shows a heatmap illustrating the results of one-pot multiplex detection of the N gene and the D614G mutation. The concentration of the target RNA was 10 nM, and only when the D614G mutation was present, a malachite green aptamer was formed and a fluorescence signal (red) was generated, demonstrating the possibility of multiplex analysis (FIG. 17C).

In a multiplex experiment in which a STAR probe for the N gene and a STAR probe for D614G were simultaneously present, the sensitivity to both the N gene and D614G of SARS-CoV-2 was evaluated. It was confirmed that targets for the N gene and D614G could be analyzed simultaneously up to 100 fM (FIG. 17D).

Therefore, it was confirmed that multiplex detection of different target nucleic acid sequences may be performed sensitively and without interference, using the STAR probe of the present invention.

### Example 5

### System for Detecting Pathogens

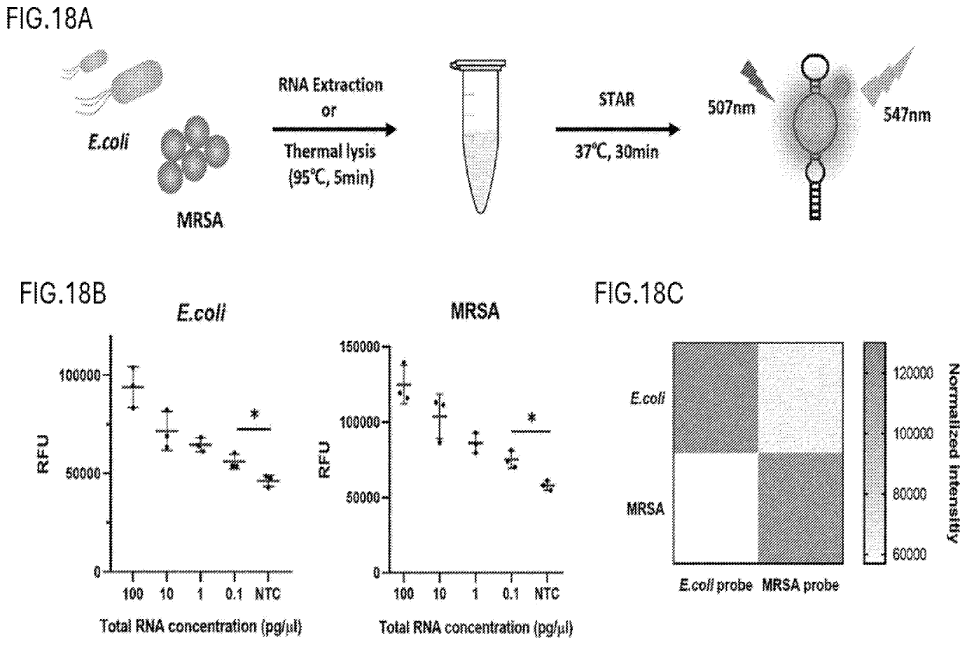

To determine whether the STAR assay may be used for a variety of applications, an experiment was performed to detect two pathogenic bacteria, E. coli (Escherichia coli) and S. aureus (Staphylococcus aureus) (FIGS. 18A-18C). For sensitive and specific detection, 16S rRNA, which is present in a large quantity in pathogens, was selected as a target and sequenced in another bacterial genus, and STAR probes specific for E. coli and S. aureus were designed.

As the target RNA sequences, the above-described sequences of SEQ ID NOs: 85 and 86 in [Table 3] were used, and as the sequence information of the DNA probe, the above-described sequences of SEQ ID NOs: 50 to 57 were used in [Table 1].

The experiment was conducted using two methods. One method was to detect the total RNA extracted from a pathogen, and the other method was to detect by performing a direct experiment in which an analysis was performed immediately after heat treatment of the pathogen without an additional nucleic acid extraction process (FIG. 17A).

Among the two experimental methods, the method in which extraction of nucleic acids was performed was conducted as follows. E. coli and S. aureus were first cultured in a Luria-Bertani liquid medium at 37° C. for 24 hours with shaking (150 rpm). Then, the total RNA was extracted from 1 ml of the medium using a bacterial RNA extraction kit.

In the direct method in which no additional nucleic acid extraction process was performed, a bacterial pellet was centrifuged at 5000 rpm, resuspended in DEPC-treated water, and heated and dissolved at 95° C. for five minutes. 4 μL of the lysate was used for the STAR analysis according to the above-described procedure.

As shown in FIG. 18B, it was confirmed that both E. coli and MRSA specifically bound to each probe, thereby generating a high fluorescence signal. The extracted total RNA was quantitatively measured at a concentration ranging from 100 pg/μL to 100 fg/μL, and it was confirmed that detection was possible even at a concentration of 100 fg/μL.

In addition, direct STAR was performed using pathogens at a concentration of 10³ cells/μL. A lysate of each pathogen (E. coli and S. aureus) was prepared by heating at 95° C. for five minutes, and then STAR was performed with E. coli and S. aureus-specific STAR probes. FIG. 18C shows a heatmap illustrating the results of direct STAR for detection of pathogenic bacteria. The concentration of each pathogen was 10³ cells/μL.

As a result, it was shown that the method of the present invention may be used to detect not only viruses but also various biomarkers, and detection is possible directly by heat treatment without pretreatment. Therefore, when the present invention is used, multiplex detection may be performed and a direct system may be established with only one enzyme in one tube, and the present invention may be used to detect pathogenic bacteria simply and quickly.

1. An isothermal one-pot reaction probe set for detecting one or more target molecules, comprising a first probe and a second probe,
wherein the first probe is a promoter probe (PP) having a structure including General Formulas I and II below, 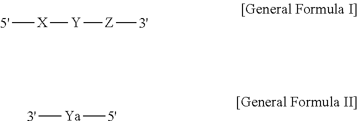
wherein in General Formula I, X is an aptamer sequence portion having an interactive labeling system including one label or multiple labels generating a detectable signal;
Y is a sequence complementary to a T7 promoter;
Z is a portion that binds or connects to a target molecule; and
X and Y are deoxyribonucleotides; and
in General Formula II, Ya is a partial sequence of the T7 promoter; and Ya is a deoxyribonucleotide;
wherein the second probe is a PP having a structure of General Formula III below, 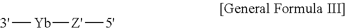
wherein in General Formula III,
Yb is absent or a partial sequence of the T7 promoter and is a deoxyribonucleotide; and
Z′ is a portion that binds or connects to a target molecule;
wherein the first probe and the second probe bind or connect to a target molecule and then transcription is initiated by a polymerase to generate a signal.

2. The probe set according to claim 1, wherein the target molecule is one or more selected from the group consisting of a nucleic acid, a protein, a cell, and adenosine triphosphate (ATP),
wherein when the target molecule is a nucleic acid, Z and Z′ are nucleic acid sequences that complementarily bind to a nucleic acid to be detected,
when the target molecule is a protein, Z and Z′ are antibodies or aptamers that are capable of binding to the protein,
when the target molecule is a cell, Z and Z′ are antibodies or aptamers that are capable of binding to one or more selected from the group consisting of extracellular proteins, cellular phospholipids, bacterial peptidoglycans, and lipopolysaccharide (LPS), and
when the target molecule is ATP, Z and Z′ are split aptamers that are capable of binding to ATP.

3. (canceled)

4. The probe set according to claim 1, wherein the Ya and Yb are partial sequences of the T7 promoter,
and when Ya and Yb are sequentially arranged in the order of Ya and Yb, the Ya and Yb form the T7 promoter, and
a Ya:Yb split ratio is 20:0 to 15:5.

5. (canceled)

6. (canceled)

7. The probe set according to claim 1, wherein General Formula I of the first probe may be modified into General Formula I′ further including an overlapping sequence W between Y and Z; 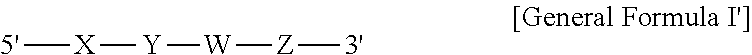
and General Formula III of the second probe may be modified into General Formula III′, further including an overlapping sequence W′ between Yb and Z′;

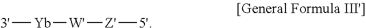

8. The probe set according to claim 7, wherein the length of overlapping sequences W and W′ is 1 bp or 2 bp.

9. (canceled)

10. The probe set according to claim 1, wherein the probe set detects circular RNA separately from linear RNA.

11. (canceled)

12. The probe set according to claim 1, wherein the isothermal one-pot reaction is performed in a unified and simultaneous manner with a one-pot reaction solution containing Tris-HCl, MgCl₂, dithiothreitol (DTT), spermidine, ribonucleotide triphosphates (rNTPs), an RNase inhibitor, and a single-stranded DNA binding protein (SSB).

13. A composition for detecting a target molecule, comprising the isothermal one-pot reaction probe set for detecting a target molecule including a first probe and a second probe of claim 1.

14. The composition according to claim 13, wherein the composition includes two or more types of isothermal one-pot reaction probe sets for detecting two or more types of target molecules.

15. The composition according to claim 14, wherein the two or more types of isothermal one-pot reaction probe sets for detecting target molecules each include different interactive labeling systems, and the two or more types of probe sets respectively bind to different target molecules to enable multiplex detection of different target molecules.

16. (canceled)

17. A kit for detecting a target molecule, comprising the composition for detecting a target molecule according to claim 13, a polymerase, and an isothermal one-pot reaction solution.

18. A method of detecting a target molecule, using the isothermal one-pot reaction probe set for detecting a target molecule of claim 1.

19. The method according to claim 18, comprising a step of performing an isothermal nucleic acid amplification reaction prior to the method of detecting the target molecule.

20. The method according to claim 19, wherein the isothermal nucleic acid amplification reaction is recombinase polymerase amplification (RPA).

21. An on-site molecular diagnostic method using the isothermal one-pot reaction probe set for detecting a target molecule according to claim 1.

22. The on-site molecular diagnostic method according to claim 21, wherein the molecular diagnostic method is used in pathogenic microorganism detection.

23. (canceled)

24. An isothermal one-pot reaction probe set for detecting SARS-CoV-2 and/or detecting a mutant thereof, comprising a third probe and a fourth probe,
wherein the third probe is a PP having a structure including General Formulas IV and V below, 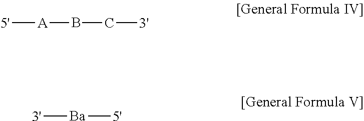
wherein in General Formula IV, A is an aptamer sequence portion having an interactive labeling system including one label or multiple labels generating a detectable signal;
B is a sequence complementary to a T7 promoter;
C is an upstream hybridization sequence (UHS) portion having a hybridization sequence complementary to a nucleic acid sequence of SARS-CoV-2 and/or a mutant thereof; the nucleic acid sequence of SARS-CoV-2 and/or a mutant thereof is DNA or RNA; and
A, B and C are deoxyribonucleotides; and
in General Formula V, Ba is a partial sequence of the T7 promoter; and Ba is a deoxyribonucleotide;
wherein the fourth probe is a PP having a structure consisting of General Formula VI, 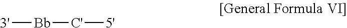
wherein in General Formula VI, Bb is absent or a partial sequence of the T7 promoter;
C′ is a downstream hybridization sequence (DHS) portion having a hybridization sequence complementary to a nucleic acid sequence of SARS-CoV-2 and/or a mutant thereof; the nucleic acid sequence of SARS-CoV-2 and/or a mutant thereof is DNA or RNA; and
Bb and C′ are deoxyribonucleotides;
wherein the third probe and the fourth probe are hybridized to a nucleic acid sequence of SARS-CoV-2 and/or a mutant thereof and then transcription is initiated by a polymerase to generate a signal.

25.-32. (canceled)

33. The probe set according to claim 24, wherein a region of the nucleic acid sequence of SARS-CoV-2 and/or a mutant thereof is an N gene or an S gene.

34. The probe set according to claim 33, wherein the isothermal one-pot reaction probe set for detecting SARS-CoV-2 specifically binds to the N gene and includes at least one probe set selected from the group consisting of a probe set in which the third probe is SEQ ID NO: 37 and the fourth probe is SEQ ID NO: 38 and a probe set in which the third probe is SEQ ID NO: 41 and the fourth probe is SEQ ID NO: 42.

35. The probe set according to claim 24, wherein the isothermal one-pot reaction probe set for detecting a mutant specifically binds to the S gene and includes a probe set in which the third probe is SEQ ID NO: 45 and the fourth probe is SEQ ID NO: 49.

36.-41. (canceled)
